(19) United States
(12) Patent Application Publication
(10) Pub. No.: US 2022/0002162 A1
(43) Pub. Date: Jan. 6, 2022

(54) METHODS AND SYSTEMS FOR PROMOTING FORMATION OF CO2 CLATHRATE HYDRATES BY THE USE OF MAGNESIUM AND OTHER ACTIVE METALS

(71) Applicants: Board of Regents, The University of Texas System, Austin, TX (US); ExxonMobil Research and Engineering Company, Annandale, NJ (US)

(72) Inventors: Aritra Kar, Austin, TX (US); Palash Acharya, Austin, TX (US); Vaibhav Bahadur, Austin, TX (US); Awan Bhati, Austin, TX (US); Ashish Mhadeshwar, Garnet Valley, PA (US); Timothy A. Barckholtz, Whitehouse Station, NJ (US)

(21) Appl. No.: 17/366,542

(22) Filed: Jul. 2, 2021

Related U.S. Application Data
(60) Provisional application No. 63/047,786, filed on Jul. 2, 2020.

(51) Int. Cl. C01B 32/55; B01J 3/03; B01J 3/00; B01J 3/02; C07D 307/06; C07C 7/20

(52) U.S. Cl. CPC C01B 32/55; B01J 3/03; B01J 3/002; C25B 1/04; C07D 307/06; C07C 7/20; B01J 3/02

(57) ABSTRACT

Described herein are methods, systems, and techniques relating to clathrate hydrate formation processes and, particularly, involving reactive metal nucleation substrates for promoting clathrate hydrate formation. The disclosed methods, systems, and techniques allow for improved nucleation rate and yield of clathrate hydrates. In some cases, the disclosed methods, systems, and techniques can also improve or reduce the amount of time needed for obtaining a given quantity of clathrate hydrate phase, for example, in desalination, gas separation and/or gas sequestration processes. The reactive metal nucleation substrate may include reactive metals from Group II, Group I, or Group XIII of the periodic table, for example, in alloyed form with other metals and/or nonmetal elements.

### CROSS-REFERENCE TO RELATED APPLICATIONS

This application claims the benefit of and priority to U.S. Provisional Application No. 63/047,786, filed on Jul. 2, 2020, which is hereby incorporated by reference in its entirety.

### FIELD

This invention is in the field of clathrate hydrate formation processes. This invention relates generally to systems, methods, and techniques for promoting nucleation and formation of clathrate hydrates in aqueous solutions.

### BACKGROUND

Clathrate hydrates are ice-like solids consisting of a lattice of hydrogen-bonded water molecules encapsulating a guest molecule. In cases where a small non-polar molecule is a gas dissolved in the water, for example, the gas molecules in solution may cause the water molecules to organize as a “host” around the gas molecule “guest” in a hydrogen bonded polyhedral crystalline phase. Clathrate hydrates can form bulk phases that visually resemble water-ice and that contain significant quantities of trapped gas. Typically, formation conditions for clathrate hydrates include elevated pressures and reduced temperatures relative to ambient atmospheric conditions (akin to those observed in sea-floor environments). Furthermore, clathrate hydrate formation is sometimes initiated by thermodynamically driven nucleation at high energy surface sites, for example, surface asperities in the inner walls of natural gas pipelines in sub-arctic regions. While several techniques for stimulating clathrate hydrate formation have been developed, kinetic rate limitations remain a consistent challenge.

### SUMMARY

Described herein are methods and systems for generating clathrate hydrates and, in particular, for promoting fast nucleation of clathrate hydrates to allow bulk formation of clathrate hydrates at appreciable rates. The disclosed methods and systems advantageously allow clathrate hydrates to form, under appropriate conditions, within minutes instead of within hours or days. Clathrate hydrates can be useful for storing large quantities of gases as guest components, which can allow these gases to be securely stored as a solid form.

In a first aspect, methods for generating clathrate hydrates are described. An example method of this aspect comprises subjecting CO₂ and a liquid comprising water to a clathrate hydrate nucleation condition while contacting the liquid with a reactive metal nucleation substrate. Such a method may be useful for generating CO₂ clathrate hydrates, for example. Example reactive metal nucleation substrates may react with the liquid to form a plurality of bubbles that facilitate nucleation of a CO₂ clathrate hydrate, such as in the liquid, at an interface between the liquid and the reactive metal nucleation substrate, or at a gas-liquid-metal interface. Useful reactive metal nucleation substrates may comprise a Group II element or an alloy thereof, or a Group I element or an alloy thereof, or a Group XIII element or an alloy thereof. In a specific embodiment, the reactive metal nucleation substrate comprises Magnesium.

The reactive metal nucleation substrate may be in any suitable form. Examples include, but are not limited to a dust, a foam, a porous scaffold, a nanostructured material, a coating, a thin film, a plate, a powder, or a felt. In some cases, it may be advantageous to use a reactive metal nucleation substrate with a high surface area. For example, a reaction between the reactive metal nucleation substrate may take place with the liquid or a component thereof, such as to generate bubbles of gas that can facilitate nucleation of the clathrate hydrate. Optionally, the reactive metal nucleation substrate comprises a plurality of particles having a diameter of from 100 nm to 100 μm. For example, the plurality of particles may be present as a colloidal suspension in the liquid. Optionally, the reactive metal nucleation substrate comprises a scaffold including a plurality of particles, such as a scaffold that includes a void volume such that the liquid flows through the void volume and introduces a plurality of gas bubbles into the liquid.

Advantageously, the methods of this aspect may be used to generate clathrate hydrates using water or a water-based liquid. For example, the liquid may be or comprise at least one of sea water, fresh water, processed water, produced water, purified water, brackish water, hypersaline water, brine, or water including an ion concentration (optionally exclusive of H⁺ ions and OH⁻ ions) or salt concentration in a range from 0% to 35% by weight, such as from 0% to 1%, from 1% to 3.5%, from 3.5% to 10%, from 10% to 15%, from 15% to 20%, from 20% to 25%, from 25% to 30%, or from 30% to 35%.

Molecules or atoms of a gas, such as CO₂, may be captured or hosted by the clathrate hydrate, which can allow for significant amounts of the gas atoms to be present in a solid hydrate form at reasonable temperatures, such as temperatures at, about equal to, or above the freezing temperature of water, though formed clathrate hydrates can be cooled to below the freezing temperature of water. As an example, CO₂ that is subjected to clathrate hydrate nucleation conditions with the liquid may comprise at least one of gaseous CO₂, liquid CO₂, or dissolved CO₂. The CO₂ may be pure or include impurities. In some cases, the CO₂ may have a purity of at least 80%. In some cases, the CO₂ may be sparged, bubbled, or sprayed into the liquid. Optionally, a method of this aspect further comprises generating the CO₂ by way of a chemical reaction.

It will be appreciated that the amount of CO₂ that may be dissolved or present in water may be a function of pH (i.e., a concentration of Hydrogen or Hydronium ions). Example pH for the liquid may be in a range of about 5 to 9. The presence of CO₂ in the liquid may impact the pH, though in some cases, the pH can be controlled, such as by addition of Hydrogen or Hydronium ions or hydroxide ions, to control the amount of CO₂ dissolved in the liquid. In some cases, it may be desirable for CO₂ to be present in as large amount as possible in the liquid, such as up to a saturation limit for CO₂ in the liquid. In some cases, the liquid may comprise a dissolved CO₂ concentration of from 0 mol/L to 0.15 mol/L.

Optionally, methods of this aspect may further comprise introducing at least one of an additive gas or an additive liquid into the liquid contacting the reactive metal nucleation substrate. For example, the additive gas or the additive liquid may comprise CO₂ or a mixture including CO₂. Optionally, the additive gas or the additive liquid may comprise a promoter for hydrate formation. Optionally, the additive liquid may comprise a surfactant or an enzyme. Optionally, introducing the additive gas may comprise bubbling the additive gas in the liquid. Optionally, introducing the additive liquid may comprise spraying an additive liquid into the liquid contacting the reactive metal nucleation substrate. The additive gas or the additive liquid may or may not become a part of the clathrate hydrate.

The clathrate hydrate nucleation conditions may generally include low temperature and high pressure. For example, the clathrate hydrate nucleation condition may comprise a pressure of greater than 150 psig or from 150 psig to 4500 psig. As another example, the clathrate hydrate nucleation condition may comprise a temperature of from 248 K to 298 K. It will be appreciated that clathrate hydrates can form at temperatures near the freezing temperature of water, and can form at different temperatures, depending on the pressure conditions, feed composition (e.g., gas or liquid mixtures), and/or liquid composition (e.g., presence of salts or promoters in water).

In some cases, subjecting the liquid and the CO₂ to the clathrate hydrate nucleation condition may occur in a pressure vessel or at a large depth (e.g., subsurface depth or sea-floor depth). For use of a pressure vessel, some methods of this aspect may further comprise maintaining the CO₂ and the liquid at the clathrate hydrate nucleation condition using a pressure controller in communication with the pressure vessel. Methods of this aspect may comprise subjecting the CO₂ and the liquid to the clathrate hydrate nucleation condition by removing heat through direct or indirect contact with a heat exchanger. Methods of this aspect may comprise maintaining the CO₂ and the liquid at the clathrate hydrate nucleation condition using a temperature controller in communication with the heat exchanger.

Advantageously, methods of this aspect may allow for nucleation of clathrate hydrates to occur in very short time scales and considerably quicker than by other methods. In some cases using conventional techniques, clathrate hydrate nucleation occurs only over very long time scales, such as greater than 24 hours. For the techniques described herein, in some cases, clathrate hydrate nucleation may occur within less than 8 hours. In some cases, clathrate hydrate nucleation may occur within less than 4 hours. Advantageously, methods of this aspect may allow for clathrate hydrate nucleation to occur in a time period of from about 1 minute to about 12 minutes. In some cases, clathrate hydrate nucleation may occur in less than 8 minutes. As used herein, occurrence in less than 8 minutes may indicate that clathrate hydrate nucleation has begun within 8 minutes from the time of application of the clathrate hydrate nucleation conditions, such as a sufficient pressure (e.g., greater than 150 psig), a sufficient temperature (e.g., less than 298 K or less than 275 K), and/or from introduction of the reactive metal substrate into the liquid.

Without wishing to be bound by any theory, the generation of bubbles by reaction of the reactive metal nucleation substrate with the liquid may, in part, facilitate nucleation of the clathrate hydrates. For example, a reaction product gas, such as Hydrogen gas (H₂), may be generated upon reaction of the reactive metal nucleation substrate with the liquid. In some cases, the plurality of bubbles may have a diameter of less than 500 m or from 10 nm to 5 mm. In some cases, insoluble and/or soluble materials that are generated upon reaction of the reactive metal nucleation substrate with the liquid may, in part, facilitate nucleation of the clathrate hydrates. For example, metal salts and/or metal hydroxides may be generated upon reaction of the reactive metal nucleation substrate with the liquid and/or with the gas. Increasing or inducing convection in the liquid, for example through circulation of bubbles or liquid, may be useful for increasing a clathrate hydrate formation rate. Clathrate hydrates may be separated from the residual liquid and gas upon nucleation using density difference and/or mechanical methods, such as to facilitate formation of additional clathrate hydrates.

Although the above discussion has focused on nucleation or formation of CO₂ clathrate hydrates, it will be appreciated that nucleation of other clathrate hydrates can be facilitated using the methods disclosed herein. For example, in some embodiments, a method for nucleation of clathrate hydrates may comprise subjecting a compound and liquid comprising water to a clathrate hydrate nucleation condition while forming a plurality of bubbles that facilitate nucleation of a clathrate hydrate comprising water and the compound. Optionally, the compound may be in a gaseous state, a liquid state, or is dissolved in the liquid. The compound and the liquid may be maintained at the clathrate hydrate nucleation condition for a period of time until an onset of clathrate hydrate nucleation. The compound may be pure or include impurities. In some cases, the compound may have a purity of at least 80%. The period of time may be less than 8 minutes. In some cases, the period of time may be from about 1 minutes to about 12 minutes. The compound may comprise, for example, at least one of CO₂, H₂S, methane, ethane, propane, butane, hydrogen, tetrahydrofuran, or cyclopentane.

Formation of the plurality of bubbles may be facilitated or occur upon contacting the liquid with a reactive metal nucleation substrate, such as comprising a Group II element or an alloy thereof, or a Group I element or an alloy thereof, or a Group XIII element or an alloy thereof.

Systems for generating clathrate hydrates are also described, in another aspect. A system of this aspect, for example for generating CO₂ clathrate hydrates, comprises a vessel comprising a reservoir for subjecting CO₂ and a liquid comprising water to a clathrate hydrate nucleation condition; and a reactive metal nucleation substrate in contact with the liquid, the reactive metal substrate reactive with the liquid to form a plurality of bubbles for nucleating formation of a CO₂ clathrate hydrate. Again, the reactive metal nucleation substrate may comprise a Group II element or an alloy thereof, or a Group I element or an alloy thereof, or a Group XIII element, such as Aluminum, Gallium, or an alloy thereof. Optionally, the reactive metal nucleation substrate comprises Magnesium. Optionally, the CO₂ comprises at least one of gaseous CO₂, liquid CO₂, or dissolved CO₂.

Optionally, the system further comprises a pump in fluid communication with the vessel for generating a pressure in the vessel associated with the clathrate hydrate nucleation condition. Optionally, the system further comprises a pressure controller in fluid communication with the vessel and in control communication with the pump for controlling the pressure in the vessel. Optionally, the system further comprises a heat exchanger in thermal communication with the vessel for generating a temperature in the vessel associated with the clathrate hydrate nucleation. As used herein, thermal communication may include direct thermal communication or indirect thermal communication. Optionally, the system further comprises a temperature controller in thermal communication with the vessel and in control communication with the heat exchanger for controlling the temperature in the vessel. Optionally, the system further comprises one or more processors; and a non-transitory computer readable storage medium in communication with the one or more processors, the non-transitory computer readable storage medium containing instructions that, when executed by the one or more processors, cause the one or more processors to perform operations. Example operations include, but are not limited to, controlling or maintaining a pressure in the vessel associated with the clathrate hydrate nucleation condition by receiving pressure sensor measurements and sending a pressure control signal to a pump in fluid communication with the vessel. Example operations include, but are not limited to, controlling or maintaining a temperature in the vessel associated with the clathrate hydrate nucleation condition by receiving temperature sensor measurements and sending a temperature control signal to a heat exchanger in thermal communication with the vessel. Example operations include, but are not limited to, generating images of formed clathrate hydrates or the interior of the clathrate hydrate nucleation vessel using an optical sensor. Optionally, the system includes an optical sensor configured to generate images of formed clathrate hydrates or an interior region of the vessel.

The vessel may be configured to maintain the liquid and the gas-phase CO₂ or CO₂ dissolved in the liquid at the clathrate hydrate nucleation condition for a period of time at least until an onset of CO₂ clathrate hydrate formation. For example, the period of time may be less than 8 minutes. In other examples, the period of time may be from about 1 minute to about 12 minutes. Although CO₂ is used as an example guest compound, systems disclosed herein can be useful with other guest compounds, such as, but not limited to, methane, ethane, propane, butane, hydrogen, tetrahydrofuran, or cyclopentane

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 provides a diagram illustrating an example system for forming clathrate hydrates, in accordance with some embodiments of the present disclosure.

FIG. 2 provides a diagram illustrating an example technique for formation of clathrate hydrates including a nucleation substrate, in accordance with some embodiments of the present disclosure.

FIG. 3A provides a photograph of a reactive metal nucleation substrate immersed in a liquid. FIG. 3B provides a photograph of a reactive metal nucleation substrate immersed in a liquid with clathrate hydrates shown.

FIG. 4 provides an overview of an example method for forming clathrate hydrates, in accordance with some embodiments of the present disclosure.

FIG. 5 provides an overview of another example method for forming clathrate hydrates, in accordance with some embodiments of the present disclosure.

FIG. 6 provides a diagram illustrating an example system for forming clathrate hydrates, in accordance with some embodiments of the present disclosure.

FIG. 7 provides a photograph of a water droplet conversion to CO₂ hydrate.

FIG. 8 provides a histogram showing fraction of droplets nucleating in various time intervals for three different droplet volumes.

FIG. 9 provides photographs of CO₂ hydrate nucleation at an Al-water interface.

FIG. 10 provides a data plot describing cumulative probability distributions for 3 different droplet volumes (10, 20 and 40 μL).

FIG. 11 provides a histogram data plot describing a fraction of 20 μL droplets nucleating in different time intervals (droplets contain 3.5 wt. % NaCl).

FIG. 12 provides a histogram data plot describing a fraction of 20 μL droplets nucleating in different time intervals (24 hour CO₂ dissolution time).

FIG. 13A provides a photograph of two droplets of water on a stainless steel surface at nucleation conditions, where a first droplet contains Sodium dodecyl-sulfate.

FIG. 13B provides a photograph showing nucleation in the first droplet containing the Sodium dodecyl-sulfate.

FIG. 13C provides a photograph showing nucleation induced in a second droplet, caused by contact with the first droplet.

### DETAILED DESCRIPTION

Described herein are methods, systems, and techniques relating to clathrate hydrate formation processes and, particularly, involving reactive metal nucleation substrates for promoting clathrate hydrate formation. The disclosed methods, systems, and techniques can allow for improved nucleation rate and yield of clathrate hydrates, for example by promoting formation of bubbles on the surface of a reactive metal nucleation substrate. The bubbles, in turn, can act as nucleation sites for the formation of clathrate hydrates. In some cases, the disclosed methods, systems, and techniques can also improve or reduce the amount of time needed for obtaining a given quantity of clathrate hydrate phase, for example, in desalination, gas separation, gas storage, and/or gas sequestration processes. The reactive metal nucleation substrate may include reactive metals from Group II, Group I, or Group XIII of the periodic table, for example, such as in pure or alloyed form with other metals and/or nonmetal elements. Advantageously, targeting the formation of clathrate hydrates at a gas-liquid and/or gas-liquid-solid interface can permit increased formation rate and yield, such as by introducing additional techniques for generating bubbles in a bulk liquid phase. For example, a substrate morphology, flow patterns of a liquid near a substrate surface, a substrate composition, a liquid composition, environmental conditions, or the like can be optimized.

In general the terms and phrases used herein have their art-recognized meaning, which can be found by reference to standard texts, journal references and contexts known to those skilled in the art. The following definitions are provided to clarify their specific use in the context of the invention.

“Clathrate hydrate” refers to a crystalline or semi-crystalline or amorphous solid including water molecules in a cage-like structure containing a compound within the cage-like structure.

“Guest compound” refers to a compound contained within the cage-like structure of a clathrate hydrate.

“Host liquid” refers to a liquid including water from which a clathrate hydrate phase forms under hydrate nucleation and formation conditions.

“Nucleation” refers to the generation of a seed crystal, within the host liquid and/or on a surface and/or at an interface between a solid, liquid, or gas, from which a clathrate hydrate phase forms.

“Formation” refers to the phase-change process by which a clathrate hydrate phase forms from a first crystal to generate a bulk clathrate hydrate.

“Reactive metal nucleation substrate” refers to a metal containing material that reacts with the host liquid or one or more compounds in the host liquid to form multiple nucleation sites from which a clathrate hydrate phase nucleates and forms a bulk clathrate hydrate.

“Water-ice” refers to solid-phase water (e.g., hexagonal crystalline ice), where water molecules do not form a clathrate hydrate cage structure.

“Two-phase line” refers to a point in a system where two phases meet. This may include a solid surface in contact with a gas phase or a liquid phase, for example.

“Three-phase line” refers to a point in a system where three phases meet. This may include a line where solid, liquid and gas phases meet, for example. Similarly, this may include a solid surface and a boundary between two immiscible liquid phases, for example.

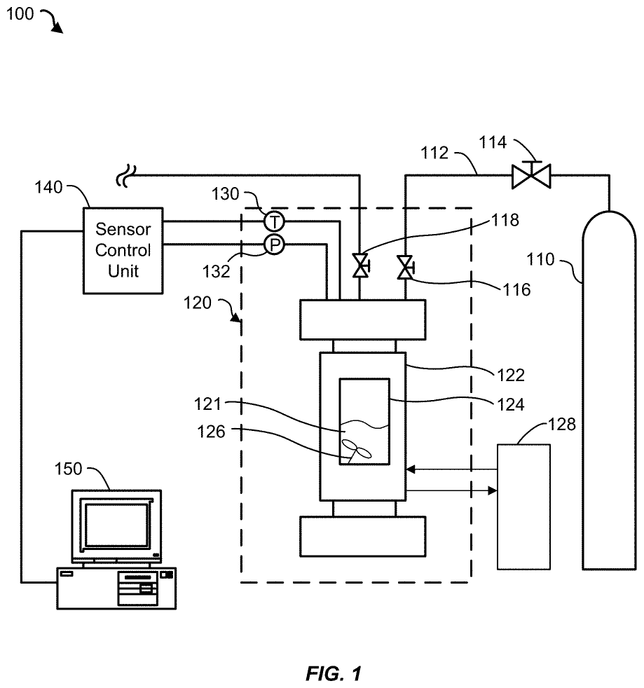

FIG. 1 provides a diagram illustrating an example system 100 for forming clathrate hydrates in accordance with an embodiment of the present disclosure. In some embodiments, the system 100 includes a hydrate guest compound 110, shown in FIG. 1 in a storage tank. The hydrate guest compound 110 may include a composition that is normally a gas at atmospheric conditions. Examples of the hydrate guest compound 110 include, but are not limited to, carbon dioxide (CO₂), methane (CH₄), ethane (C₂H₆), propane (C₃H₈), butane (C₄H₁₀), nitrogen gas (N₂), hydrogen gas (H₂), hydrogen sulfide (H₂S), NOₓ, SOₓ, or the like. In some embodiments, the hydrate guest compound 110 is generated in a combustion unit or a petrochemical unit, as a product of a combustion process or a catalytic refining process, for example. In some embodiments, the hydrate guest compound 110 is generated via chemical reaction occurring in the host liquid 121. In some embodiments, the hydrate guest compound 110 is provided for hydrate formation via an inlet 112, including controls 114 (e.g., a pressurized supply system), such that the hydrate guest compound 110 may be provided as a gas or a liquid, or both, as a function of the pressure and the temperature at which the hydrate guest compound 110 is provided. Furthermore, the hydrate guest compound 110 may be provided as a dissolved gas in a liquid at a concentration, dependent on the pressure and temperature. For example, the concentration may be a saturation concentration and/or a concentration falling in the range from about 0 mol/L to about 0.15 mol/L, such as from 0 mol/L to 0.01 mol/L, from 0.01 mol/L to 0.02 mol/L, from 0.02 mol/L to 0.03 mol/L, from 0.03 mol/L to 0.04 mol/L, from 0.04 mol/L to 0.05 mol/L, from 0.05 mol/L to 0.06 mol/L, from 0.06 mol/L to 0.07 mol/L, from 0.07 mol/L to 0.08 mol/L, from 0.08 mol/L to 0.09 mol/L, from 0.09 mol/L to 0.10 mol/L, from 0.10 mol/L to 0.11 mol/L, from 0.11 mol/L to 0.12 mol/L, from 0.12 mol/L to 0.13 mol/L, from 0.13 mol/L to 0.14 mol/L, or from 0.14 mol/L to 0.15 mol/L. In some embodiments, the hydrate guest compound 110 is pre-cooled prior to being provided to the host liquid 121, for example, by using a heat exchanger upstream of the inlet 112.

In some embodiments, the system 100 includes a formation reactor 120 for subjecting a host liquid 121 containing the hydrate guest compound 110 to conditions suitable for formation of a clathrate hydrate phase. In some embodiments, the formation reactor 120 comprises a bubble column reactor. In some embodiments, the formation reactor 120 comprises an air-lift reactor. In some embodiments, the host liquid 121 may include water having a concentration of dissolved salts. For example, the host liquid 121 may include, but is not limited to, seawater, brackish water, fresh water, processed water, produced water, purified water, hypersaline water, brine, or water including an ion concentration in a range from 0% to 35% by weight, such as from 0% to 10% by weight. In some embodiments, the salts may include sodium, potassium, calcium, other metal salts, chlorides, sulfates, etc. In some embodiments, an amount of total dissolved solids (TDS) in the host liquid 121 may be in a range from 0 to 50,000 ppm. In some embodiments, the host liquid 121 may include one or more acids including, but not limited to carbonic acid, sulfuric acid, phosphoric acid, or the like. In some embodiments, the host liquid 121 may have a pH value falling within a range of about 5 to 9. In some embodiments, the pH value may be less than 5. It will be appreciated that the amount of guest compound 110 dissolved or otherwise present in the host liquid 121 may control or impact the pH in some cases. In some cases, the amount of guest compound 110 dissolved or otherwise present in the host liquid 121 may correspond to a saturation amount.

The formation reactor 120 may include a vessel 122 for subjecting the host liquid 121 to an elevated pressure, where the vessel includes a reservoir 124 for holding the host liquid 121, a pressurizing subsystem (e.g., a compressor, a pressure cell, a piston, a pump, etc.), as well as additional component subsystems. For example, in some embodiments, the formation reactor 120 includes a circulation device 126, such as an impeller, a circulation pump, and/or other circulation device, in the reservoir 124 for stirring the host liquid 121 in the reservoir 124. In some embodiments, the reservoir 124 subjects the host liquid 121 to a formation pressure, for example, falling within a range of 150 psig to 4500 psig. Example formation pressures may be from 150 psig to 500 psig, from 500 psig to 1000 psig, from 1000 psig to 1500 psig, from 1500 psig to 2000 psig, from 2000 psig to 2500 psig, from 2500 psig to 3000 psig, from 3000 psig to 3500 psig, from 3500 psig to 4000 psig, or from 4000 psig to 4500 psig. In some embodiments, the formation pressure is greater than 150 psig. In some embodiments, the formation pressure is greater than 4500 psig. In some embodiments, the formation reactor 120 includes one or more inlets 116 and/or one or more outlets 118, for example, as a gas exchange manifold, for providing the hydrate guest compound 110 to the reservoir 124. In some embodiments, the formation reactor 120 is in thermal communication with a heat exchanger 128 for cooling the host liquid 121 and/or maintaining the host liquid 121 at a hydrate formation temperature. In some embodiments, the hydrate formation temperature falls within a range from about 248 K to about 298 K (i.e., about −25° C. to about 25° C.), such as from 248 K to 253 K, from 253 K to 258 K, from 258 K to 263 K, from 263 K to 268 K, from 268 K to 273 K, from 273 K to 274 K, from 274 K to 275 K, from 275 K to 276 K, from 276 K, to 277 K, from 277 K to 278 K, from 278 K to 283 K, from 283 K to 288 K, from 288 K to 293 K, or from 293 K to 298 K. In some embodiments, the hydrate formation temperature is less than 275 K, less than 285 K, or less than 295 K. Temperatures of at or slightly above the freezing temperature of water may be used, for example, as such temperatures may limit or prevent water-ice from forming when nucleation or formation of clathrate hydrates are desired. In some embodiments, the hydrate formation temperature is less than 273 K. Temperatures below the freezing temperature of water may be used for a limited time in some cases, for example, to accelerate the nucleation of clathrate hydrates. Once formed, clathrate hydrates can be maintained near or below a freezing temperature of water.

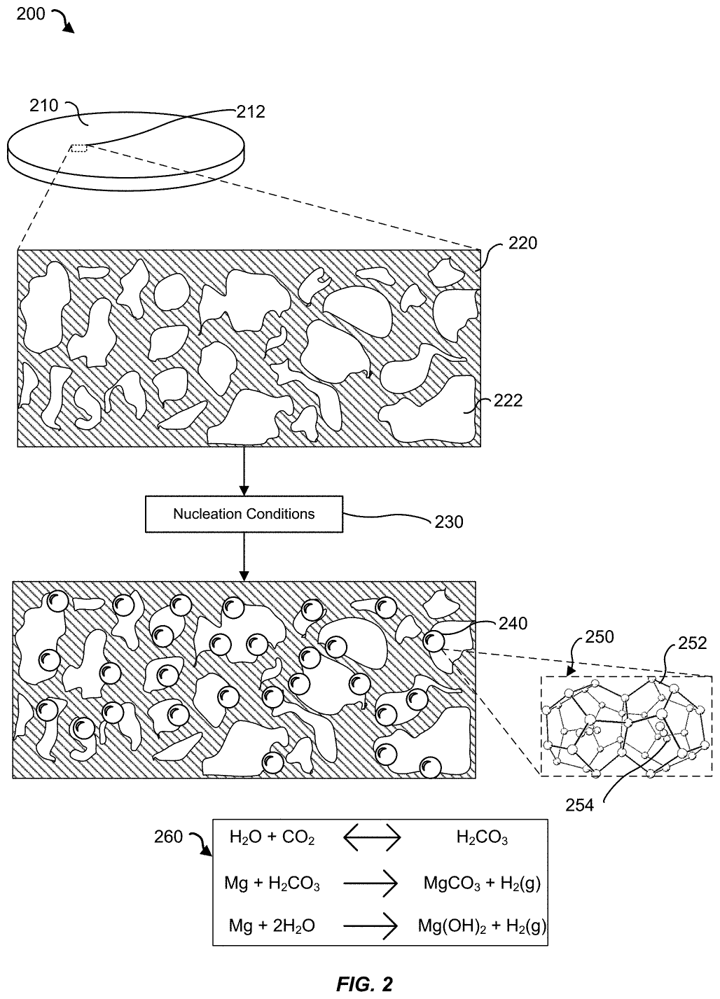

The formation reactor 120 may include liquid and/or gas exchange subsystems for maintaining chemical conditions of the host liquid 121 and for removing accumulated gases that may evolve during operation. In some embodiments, as described in more detail in reference to FIG. 2, operation of the formation reactor 120 may include generating a plurality of bubbles. Coalescence of the plurality of bubbles may act as a source of the accumulated gases. The liquid exchange may also include a separator unit to remove bulk clathrate hydrates, such that a concentrated eluent may be separated from the bulk clathrate hydrate phase, for example, as part of a desalination and/or filtration process.

In some embodiments, the formation reactor 120 includes one or more sensors including, but not limited to, a visual or optical sensor (e.g., a camera), a temperature sensor 130 and/or a pressure sensor 132, in control communication with a sensor control unit 140 for measuring a temperature and/or a pressure in the reservoir 124. In some embodiments, the sensor control unit 140 is in communication with a computer system 150 configured with computer readable instructions for operating the formation reactor 120 or components thereof. In some embodiments, the computer system 150 operates the formation reactor without user interaction (e.g., automatically). The computer system 150 may maintain the host liquid 121 at a set of formation conditions, as described in more detail in reference to FIG. 2, for a period of time during which the clathrate hydrate formation or nucleation occurs. In some embodiments, the period of time is less than eight minutes. In some embodiments, the period of time falls within a range of about 1 minute to about 12 minutes. For example, clathrate hydrates may be nucleated in from 0 minutes to 1 minute, from 1 minute to 2 minutes, from 2 minutes to 3 minutes, from 3 minutes to 4 minutes, from 4 minutes to 5 minutes, from 5 minutes to 6 minutes, from 6 minutes to 7 minutes, from 7 minutes to 8 minutes, from 8 minutes to 9 minutes, from 9 minutes to 10 minutes, from 10 minutes to 11 minutes, or from 11 minutes to 12 minutes. In some embodiments, the formation reactor 120 is first subjected to the formation temperature and subsequently the formation pressure. Alternatively, the formation reactor 120 may be subjected to the formation pressure and subsequently cooled to the formation temperature. In some cases, the formation pressure and formation temperature may be controlled or achieved simultaneously.

As described in more detail in reference to FIG. 2, a metal substrate may promote clathrate hydrate formation or nucleation, for which the formation reactor 120 may include one or more forms of the metal substrate disposed within the reservoir 124 for contacting the host liquid 121. In some embodiments, the metal substrate is suspended in the host liquid 121. Alternatively, the metal substrate may be disposed on a surface of the reservoir 124 and/or an assembly for receiving one or more metal substrates and for maintaining the one or more metal substrates in contact with the host liquid 121. In some embodiments, the assembly may generate and/or provide ultrasonic energy to the liquid near the one or more metal substrates in contact with the host liquid 121. In such cases, the ultrasonic energy may accelerate clathrate hydrate nucleation and/or formation. For example, the ultrasonic energy may stimulate nucleation and crystallization of clathrate hydrate particles and/or bulk phases. Additionally or alternatively, the ultrasonic energy may desorb accumulated gas bubbles from one or more surfaces of the one or more metal substrates in contact with the host liquid 121, such that the accumulated gas bubbles may be suspended in the host liquid 121, as described in more detail in reference to FIG. 2. In some embodiments, the assembly may be configured to expose the one or more metal substrates to a three-phase line including the host liquid 121 and the hydrate guest compound 110.

In some embodiments, the assembly may be configured to provide heat in a near-surface region of the liquid, for example, by an exothermic reaction between a reactive component of the assembly and a reactant dissolved and/or suspended in the liquid. For example, the assembly may include reduced iron nanoparticles to generate heat through exothermic oxidation. Similarly, the assembly and/or suspended metal substrates may be subjected to localized heating by radiant energy. For example, a laser or other radiant heat source (e.g., an IR light source) may be directed toward the assembly and/or liquid. Such heat may be useful for generating localized boiling and/or bubble formation and associated clathrate hydrate nucleation.

In some embodiments, an inert nucleation substrate is provided to the liquid. For example, silicon dioxide particles may be suspended in the liquid. Similarly, one or more surfaces provided with a characteristic surface roughness may be suspended in the liquid. In some cases, such inert nucleation substrates may provide controlled nucleation regions in the liquid for nucleation and growth of hydrates. For example, the characteristic root mean square (rms) surface roughness of such surfaces may be in the range of 10 nm to 100 μm.

FIG. 2 provides a schematic illustration of an example technique 200 for formation or nucleation of clathrate hydrates including a nucleation substrate in accordance with an embodiment of the present disclosure. In some embodiments, a metal substrate 210 may be used to promote nucleation and/or formation of clathrate hydrates in a liquid. In some embodiments, the metal substrate 210 includes a Group II element (e.g., Beryllium, Magnesium, Calcium, etc.) and/or an alloy including a Group II element (e.g., a Magnesium alloy). In some embodiments, the metal substrate 210 includes a Group I element (e.g., Lithium, Sodium, Potassium, etc.), such as an alloy including a Group I element. In some embodiments, the metal substrate 210 includes a Group XIII element or an alloy including a Group XIII element. For example, the Group XIII element may include Gallium, Aluminum, or an alloy containing Gallium and/or Aluminum. The metal substrate may include components other than Group I, Group II, or Group XIII elements, which may be inert or non-reactive. In some embodiments, the metal substrate 210 may include trace level of impurities.

In some embodiments, the metal substrate 210 may include at least one of a dust, a foam, a porous scaffold, a nanostructured material, a coating (e.g., a coating on another surface, for example, of a hydrate nucleation vessel), a film, a thin film, a plate, a powder, or a felt. For example, the metal substrate 210 may include a porous magnesium foam characterized by a void fraction and a plurality of surface asperities. In some embodiments, the metal substrate 210 may include a reactive region 212, for example, where the metal substrate 210 may not have uniform composition or structure to focus and/or restrict clathrate hydrate formation to one or more regions on a surface of the metal substrate. For example, the metal substrate 210 may include a ceramic, plastic, or other inert scaffold including particles of a reactive metal (e.g., magnesium) forming multiple reactive regions 212. The metal substrate 210 may further include, but is not limited to, nanostructured particles, microstructured particles, larger particles, or the like, including the reactive metal, such that the metal substrate 210 may be suspended as a colloidal suspension in a liquid containing a guest compound (e.g., host liquid 121 of FIG. 1), for example, using an impeller or other agitation and/or stirring implement (e.g., circulation device 126 of FIG. 1), or induced by gas bubbles fed into the vessel. In some embodiments, the impeller may provide gas bubbles directly by inducing cavitation in the liquid. The particles may be characterized by a particle size distribution whereby an average diameter of the particles falls within a range of 10 nm to 100 μm.

In some embodiments, the reactive region 212 may be a portion or the entire surface of the metal substrate 210 that is exposed to the liquid containing the guest compound. In some embodiments, the reactive region 212 includes a reactive surface 220 (e.g., comprising Magnesium) at which a chemical reaction occurs for promoting the clathrate hydrate formation in the liquid. While the reaction may not play a direct role in the formation of clathrate hydrates (e.g., the clathrate hydrate phase is not a reaction product of a reaction between the reactive surface 220 and the liquid containing the guest compound), the reaction may generate an intermediate phase to act as a nucleation site, as described further below.

In some embodiments, the reactive surface 220 is exposed to the liquid containing the guest compound at a plurality of boundary regions 222 (e.g., void volume, surface asperities, inert inclusions, etc.) where the boundary regions may be characterized by a higher surface energy relative to an ideal surface of the metal substrate 210. While there is not a single mechanism to which the technique 200 is constrained, chemical and/or thermodynamic processes (e.g., heterogeneous surface reactions, phase changes, crystallization, etc.) may be favored at boundaries (interfaces) and/or locations of high surface energy.

In some embodiments, the technique 200 includes subjecting the metal substrate 210 to nucleation and growth conditions 230, for example as described in more detail in reference to FIG. 1. The nucleation and growth conditions 230 may include a formation temperature and a formation pressure at which clathrate hydrate formation is possible and/or favored over formation of other solid phases (e.g., water-ice). At the nucleation and growth conditions 230, a heterogeneous surface reaction may occur at the reactive surface 220 of the metal substrate 210, producing a plurality of bubbles 240. As described above, the technique 200 may include maintaining the nucleation and growth conditions 230 for a period of time. In some cases, as with coupled chemical-thermodynamic processes, the period of time may depend on the nucleation and growth conditions 230. For example, the heterogeneous surface reaction may be characterized by a reaction rate that is a function of one or more parameters including the nucleation and growth conditions 230 (e.g., formation pressure, formation temperature, etc.). Furthermore, the technique 200 may include one or more ordered steps for applying the nucleation and growth conditions 230. For example, the technique may include cooling the metal substrate 210 to the formation temperature, followed by pressurizing the environment of the metal substrate 210 to the formation pressure. Alternatively, the ordered steps may be reversed. In some cases, cooling the metal substrate 210 to the formation temperature and pressurizing the environment of the metal substrate 210 to the formation pressure may be done simultaneously.

In some embodiments, each bubble of the plurality of bubbles 240 may act as a nucleation site or nucleation promoter for nucleation of a clathrate hydrate phase 250 including both a host compound 252 (e.g., water) and a guest compound 254 (e.g., a pure component such as CO₂ or a mixture of various molecules). The clathrate hydrate phase 250 may form at a rate that depends on the radius of a bubble, residence time of the bubble, gas solubility, gas-liquid mass transfer, local pressure, local temperature, among other parameters. The plurality of bubbles 240 may form such that the bubbles are characterized by a size distribution, whereby the bubbles may have a typical diameter falling within a range of 10 nm to 5 mm. In some embodiments, the plurality of bubbles 240 may be characterized by a diameter less than 500 micrometers.

In some embodiments, the plurality of bubbles 240 include a reaction product gas. The reaction product gas may include Hydrogen gas (H₂), generated by one or more chemical reactions following one or more reactions shown in reaction scheme 260. For example, the reaction scheme 260 may include forming an acid (e.g., forming Carbonic acid from Carbon dioxide and water). The reaction scheme 260 may also include a reaction between the metal substrate 210 (e.g. Magnesium) and the acid to form a metal salt (e.g., Magnesium carbonate) and an evolved gas (e.g., Hydrogen gas). The reaction scheme 260 may also or alternatively include a reaction between the metal substrate 210 and water to form a metal salt (e.g., a metal hydroxide) and an evolved gas (e.g., Hydrogen gas). Additionally or alternatively, the plurality of bubbles 240 may be generated in whole or in part using a guest compound and/or a different gas introduced through a gas inlet system (e.g., inlet 116 of FIG. 1). For example, a sparger or other gas inlet technique for generating individual bubbles of the characteristic diameter may be used to generate bubbles of the guest compound (e.g., CO₂). Additionally or alternatively, Hydrogen gas may be introduced directly to the liquid in this manner.

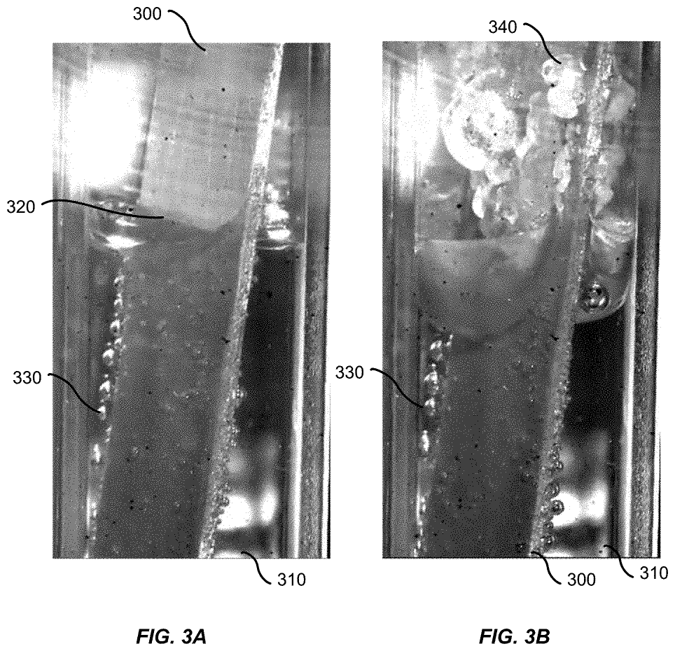

FIG. 3A provides a photograph of a reactive metal nucleation substrate submerged in a host liquid in an atmosphere including a guest compound, in accordance with an embodiment of the present invention. For example, a reactive metal nucleation substrate 300 (e.g., a Magnesium alloy, Mg AZ31B) is submerged or partially submerged in a host liquid 310 (e.g., de-ionized water), in an environment containing a guest compound (e.g., guest compound 110 of FIG. 1). In FIG. 3A, the reactive metal nucleation substrate 300 is partially submerged in the host liquid 310, such that a three-phase line 320 is formed at the interface between the host liquid 310, the reactive metal nucleation substrate 300, and the environment containing the guest compound. At the three-phase line 320, the reactive metal nucleation substrate 300 may be in contact with both the host liquid 310 and a saturated concentration of the guest compound. In some embodiments, a plurality of bubbles 330 form on a submerged surface of the reactive metal nucleation substrate 300, as described above.

FIG. 3B shows a photograph of a reactive metal nucleation substrate 300 submerged in a host liquid 310 in an atmosphere including a guest compound and a clathrate hydrate phase 340, in accordance with embodiments of the present invention. In some embodiments, for example, when a three-phase line is formed between the host liquid 310, the reactive metal nucleation substrate 300, and the environment containing the guest compound, a clathrate hydrate phase 340 may nucleate and form. In some embodiments, the clathrate hydrate phase 340 forms in the host liquid, below the three-phase line 320. In some embodiments, the clathrate hydrate phase 340 forms above the three-phase line 320 directly on a surface of the reactive metal nucleation substrate 300. Optionally, capillary action may draw liquid onto the clathrate hydrate phase 340 as it forms, allowing the clathrate hydrate phase 340 to form in substantial amounts above the three-phase line 320. Optionally, water vapor present above the three-phase line 320 may be converted into the clathrate hydrate phase 340.

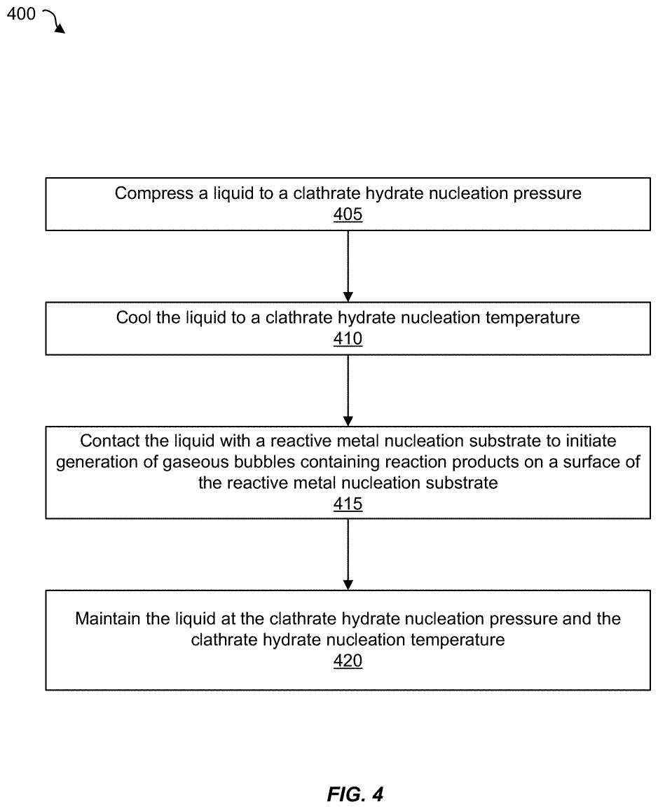

FIG. 4 provides an overview of an example method 400 for forming the clathrate hydrates. At block 405, a liquid is compressed to a clathrate hydrate nucleation pressure. For example, the liquid may comprise water and the nucleation pressure may be in excess of 150 psi. The liquid may optionally include a compound for inclusion in the clathrate hydrate as a guest, such as CO₂.

At block 410, the liquid is cooled to a clathrate hydrate nucleation temperature. The nucleation temperature may be close to or about 0° C., such as from −5° C. to 5° C. It may be useful for the nucleation temperature to be greater than the freezing temperature of water, so as to limit or prevent formation of water-ice and allow preferential nucleation and formation of a clathrate hydrate. The order of blocks 405 and 410 may be reversed, such that the liquid is cooled then pressurized. Alternatively, blocks 405 and 410 may be combined, such that the liquid is cooled and pressurized simultaneously.

At block 415, the liquid is contacted with a reactive metal nucleation substrate which may initiate generation of gaseous bubbles containing reaction products (e.g., Hydrogen gas) on a surface of the reactive metal nucleation substrate. The reactive metal nucleation substrate may comprise a Group I element, a Group II element, a Group XIII element or alloys comprising at least one Group I element, Group II element, or Group XIII element. In some cases, the reactive metal nucleation substrate may comprise magnesium or an alloy thereof.

At block 420, the liquid is maintained at the nucleation temperature and nucleation pressure, such as for an amount of time sufficient for nucleation and growth of the clathrate hydrate. The contact between the liquid and the reactive metal nucleation substrate may result in prompt nucleation of the clathrate hydrates, such as by way of the gas bubbles, at least in part. In some cases, the amount of time may be as short as a few minutes, such as 1 minute or less, 2 minutes or less, 3 minutes or less, 4 minutes or less, 5 minutes or less, 6 minutes or less, 7 minutes or less, 8 minutes or less, 9 minutes or less, 10 minutes or less, 11 minutes or less, or 12 minutes or less.

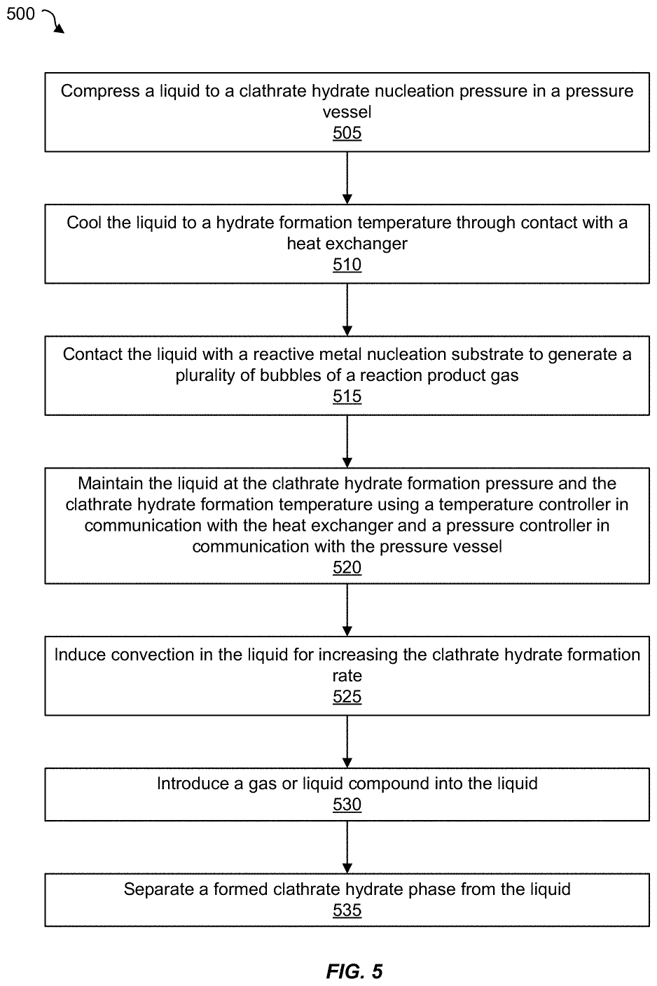

FIG. 5 provides an overview of another example method 500 for forming clathrate hydrates. At block 505, a liquid is compressed to a clathrate hydrate nucleation pressure in a pressure vessel. The pressure vessel may be constructed so as to permit the generation of high pressures within, such as pressures in excess of 150 psi or up to 4500 psi, or more. The compression of the liquid may occur via use of one or more pumps, pressure sensors, pressure controllers, or the like. The liquid may optionally include a compound for inclusion in the clathrate hydrate as a guest, such as CO₂. As described in more detail below, in some embodiments, the liquid is pressurized following cooling and a rest period.

At block 510, the liquid is cooled to a clathrate hydrate nucleation temperature through contact with a heat exchanger. The heat exchanger may facilitate removal of heat from the liquid to lower the temperature of the liquid. The nucleation temperature may be close to or about 0° C., such as from −25° C. to 25° C. It may be useful for the nucleation temperature to be greater than the freezing temperature of water, so as to limit or prevent formation of water-ice and instead allow preferential nucleation and formation of a clathrate hydrate. Cooling of the liquid may occur via use of one or more temperature sensors, temperature controllers, or the like. In some embodiments, cooling the pressure vessel may include cooling the pressure vessel for a set duration of time (e.g., 1-30 minutes) such that the liquid reaches a target temperature. For example, the pressure vessel may be cooled at −15° C. for 20 minutes to reach a target temperature of approximately 1° C. As described in more detail below, in some embodiments, cooling is preceded by purging the pressure vessel. The order of blocks 505 and 510 may be reversed, such that the liquid is cooled then pressurized. Alternatively, blocks 505 and 510 may be combined, such that the liquid is cooled and pressurized simultaneously.

At block 515, the liquid is contacted with a reactive metal nucleation substrate which may initiate generation of gaseous bubbles containing reaction products (e.g., Hydrogen gas) on a surface of the reactive metal nucleation substrate. The reactive metal nucleation substrate may comprise a Group I element, a Group II element, a Group XIII element, or alloys comprising at least one Group I element, Group II element, or Group XIII element. In some cases, the reactive metal nucleation substrate may comprise Magnesium or an alloy thereof.

At block 520, the liquid is maintained at the nucleation temperature and nucleation pressure, such as for an amount of time sufficient for nucleation and growth of the clathrate hydrate, using a temperature controller and a pressure controller. In some cases, a temperature sensor or a pressure sensor may be positioned in thermal or fluid communication with the pressure vessel to allow for determination of the temperature and/or pressure therein in real-time. The contact between the liquid and the reactive metal nucleation may result in prompt nucleation of the clathrate hydrates, such as by way of the gas bubbles. In some cases, the amount of time may be as short as a few minutes, such as 1 minute or less, 2 minutes or less, 3 minutes or less, 4 minutes or less, 5 minutes or less, 6 minutes or less, 7 minutes or less, 8 minutes or less, 9 minutes or less, 10 minutes or less, 11 minutes or less, or 12 minutes or less.

At block 525, convection within the liquid may be optionally induced to facilitate or increase a rate of formation of the clathrate hydrate. For example, in some cases, stirring the liquid may allow for the formation rate of the clathrate hydrate to increase as compared to not stirring the liquid.

At block 530, a gas or liquid compound may be optionally introduced in the liquid or above the liquid. For example, as the clathrate hydrate forms, the liquid may become depleted from the compound, at least in part, so introducing additional amounts of the compound may be useful for maintaining a concentration of the compound in the liquid. In some cases, the gas or liquid compound may be introduced at a concentration in aqueous solution to reduce water depletion during the formation and growth of clathrate hydrates.

At block 535, the formed clathrate hydrate may be separated from the liquid.

In some embodiments, one or more blocks of the method 500 may be reordered and/or omitted, such that the method 500 may proceed according to a different arrangement. Furthermore, at one or more points of the method 500, additional blocks may be added or timing elements may be added. In some embodiments, for example, the method 500 may include purging the pressure vessel. Purging the pressure vessel may be undertaken at a pressure including, but not limited to, 5 PSIG, 10 PSIG, 15 PSIG, 20 PSIG, 25 PSIG, 30 PSIG and increments therein. Purging the pressure vessel may be undertaken for a duration of time including, but not limited to, 10 sec, 15 sec, 20 sec, 25 sec, 30 sec, 35 sec, 40 sec, 45 sec, 50 sec, 55 sec, 60 sec, and increments therein. Purging the pressure vessel may be undertaken using the guest gas as a purge gas (e.g., using CO₂).

In some embodiments, the method 500 includes resting the liquid before pressurizing the pressure vessel to the nucleation pressure. For example, a temperature controller may maintain the liquid temperature at the target temperature for a rest period prior to pressurizing the liquid. In some embodiments, the rest period may include, but is not limited to, a duration of 10 min, 20 min, 30 min, 40 min, 50 min, 60 min, 70 min, 80 min, 90 min, 100 min, 110 min, 120 min, and increments therein.

The invention may be further understood by the following non-limiting examples.

### Example 1: Magnesium-Based Promotion of Nucleation of Carbon Dioxide Hydrates

Gas hydrate formation has several applications in CO₂ sequestration, flow assurance and desalination. Nucleation of hydrates is often constrained by very high induction (wait) times, which necessitates the use of complex nucleation promotion techniques to form hydrates. A simple, passive nucleation promotion technique is described in this example, wherein a Magnesium surface significantly accelerates nucleation of CO₂ hydrates. Measurements of induction times for the CO₂ hydrate nucleation were undertaken using water droplets as individual micro-systems for hydrate formation. The influence of various metal surfaces, droplet size, CO₂ dissolution time, and presence of salts in water can impact the nucleation kinetics. In general, the Magnesium-water interface may be responsible for nucleation promotion. In particular, Hydrogen bubbles generated at the Magnesium-water interface may be responsible for nucleation promotion.

Clathrate hydrates are ice-like solids consisting of a lattice of hydrogen-bonded water molecules (host) encapsulating a guest molecule. Gas hydrates (Methane, Carbon dioxide) form under high-pressure, low-temperature conditions. Formation involves nucleation of the first ‘cluster’ of stable hydrate molecules followed by growth. Nucleation of hydrates is generally characterized by very long induction/wait times, typically ranging from hours to days, especially in a quiescent medium. This challenge can be addressed via nucleation-promoting techniques such as the use of surfactants, mechanical agitation, quaternary ammonium salts and electronucleation. This example describes how Magnesium may strongly promote nucleation of CO₂ hydrates.

Experiments were also conducted using cuvettes containing varying quantities of de-ionized water, with a magnesium (Mg) plate therein. Two configurations were used: i) partially submerged Mg plate with a three-phase contact line, and ii) a completely submerged Mg plate without a three-phase line. Baseline experiments were conducted without any plates and with a stainless steel plate as controls. In these control cases no nucleation was observed within 24 hours. In contrast, nucleation was observed on both configurations using the Mg plates. Results are shown in Table 1.

TABLE 1

| | Number of | Nucleation time | Avg. & Std. |
|---|---|---|---|
| Experiment | experiments | (minutes) | Dev. (minutes) |
| No Mg plate | 2 | >24 hours | >24 hours |
| Cuvette 1: 1.5 ml water + Mg plate | 2 | 8, 11 | 9.5 ± 1.5 |
| Cuvette 2: 1.8 ml water + Mg plate | 4 | 13, 14, 14, 14 | 13.8 ± 0.4 |
| Cuvette 3: 2.5 ml water + Mg plate | 3 | 58, 82, 112 | 84 ± 22.1 |
| (completely submerged) | | | |
| Cuvette 4: 2.8 ml water + Mg plate | 4 | 27, 32, 61, 93 | 53.3 ± 26.4 |
| (completely submerged) | | | |
| Cuvette 5: 1.5 ml water + Mg plate | 6 | 0.4, 0.5, 1, 1, 1.6, 1.7 | 1 ± 0.5 |

Results indicate two potential time-dynamics. A first time-dynamic of fewer than 15 minutes nucleation time corresponds to cases where the metal surface is exposed to the gas phase, forming a three-phase line. A second time-dynamic of nearer to an hour nucleation time corresponds to cases where the metal surface is entirely submerged in water. In both cases, nucleation time is a fraction of that for cases without a metal surface.

These results clearly highlight the influence of Magnesium in ‘catalyzing’ nucleation in a CO₂-rich water solution, independent of the gas-phase CO₂. A variety of different mechanisms may be responsible for nucleation, but this study reveals nucleation promotion as a consequence of bubbles generated due to reactions at the Mg-water interface.

Without wishing to be bound by any theory, one cause of nucleation promotion may not be directly related to the ionic species generated at or present the interface. Instead, Hydrogen (H₂) bubble generation at the surface may be responsible for nucleation. Hydrogen nano-bubbles can form at the surface where the resulting high Laplace pressure generated due to the small radii may lead to favorable conditions for the formation of hydrogen hydrates. Presently, Mg reacting with carbonic acid/water can lead to generation of H₂ bubbles, which can seed the nucleation of CO₂ hydrates. Carbonic acid can also assist in breaking down any thin or native oxide layers to promote Mg-water contact. A lack of nucleation on Cu surfaces supports this analysis. Cu is less electropositive than Mg and does not easily undergo displacement reactions to generate H₂, which would lead to bubble formation. It is noted that detecting such bubbles visually or via in situ spectroscopy is challenging since these experiments are carried out in a high-pressure cell, and the concentrations of any species will be low. While H₂ bubbles appear to be a cause of nucleation promotion, additional factors such as the roughness/texture at the micro/nano scale may also promote or contribute to nucleation.

### Example 2: Aluminum-Based Promotion of Nucleation of Carbon Dioxide Hydrates

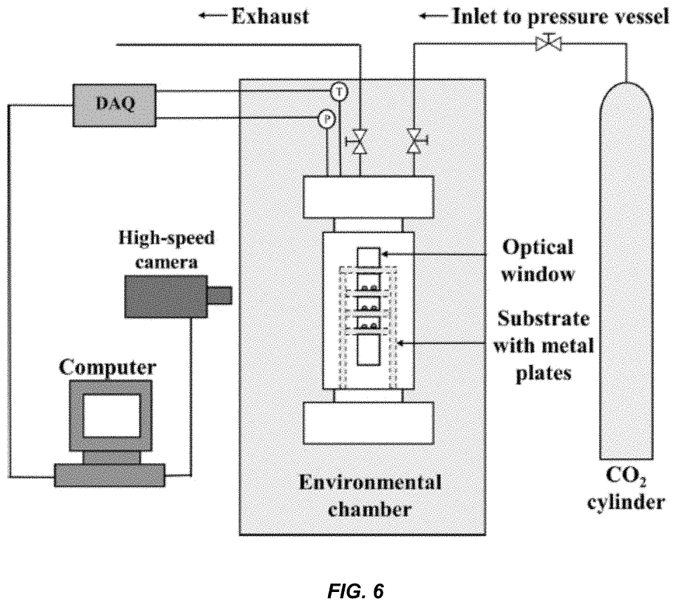

Experiments on CO₂ hydrate nucleation were conducted using water droplets in CO₂ ambient in a high pressure cell. While hydrates can form from bulk liquid-gas mixtures, the use of droplets allows conducting multiple experiments in one run. Each droplet acts as an independent system, making it possible to obtain statistically significant data, bearing in mind that nucleation is stochastic and that hydrate formation experiments are usually very long. The use of droplets/bubbles to study nucleation and formation of hydrates and ice is widely employed. This approach also enables high-quality visualization of kinetics and crystal growth. A schematic of an example experimental setup is depicted in FIG. 6. A custom-built, nonstirred, 450 mL pressure vessel with sapphire windows was used. Deionized (DI) water droplets (equal volumes unless specified otherwise) were dispensed on horizontally mounted metal plates in the vessel. New metal plates and droplets were used for every experiment to avoid the possibility of changes in surface chemistry/morphology and to avoid the memory effect. Up to three such plates (with 2-6 droplets on each) could be accommodated inside the pressure vessel in a single experiment. The pressure vessel was placed in an environmental chamber to cool it to hydrate formation temperatures. Droplets were monitored with a high speed camera fitted with a macro lens. Four surfaces were studied: aluminum, anodized aluminum, copper, and stainless steel (SS). All the metallic surfaces (Al, Cu, and SS) had a polished mirror-like texture to minimize the influence of surface roughness on nucleation promotion. The root mean square (rms) values of the surface roughness for Al, Cu, and SS plates were 40, 49, and 61 nm, respectively. The surfaces were covered to prevent contact with air; the protective covering was removed just prior to the experiments to minimize contamination and oxide formation. In summary, it involved pipetting multiple droplets onto the surface, followed by pressurization of the chamber with 99.99% purity CO₂ (3 MPa) at 20° C., and a dissolution time of 90 min (unless specified otherwise) to allow CO₂ diffusion into the water. Next, the chamber was cooled to 0.5° C.; a temperature higher than 0° C. was selected to eliminate the possibility of ice formation.

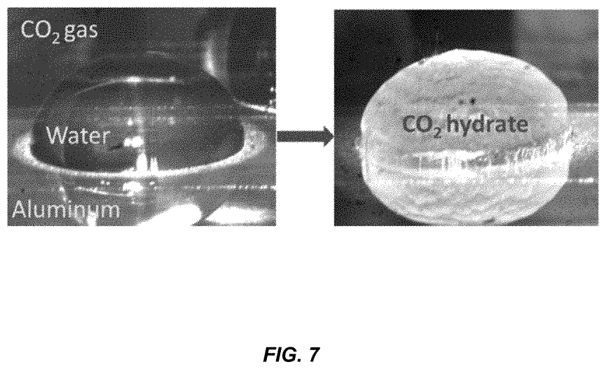

Nucleation was detected via continuous visualization; upon nucleation, the droplet turns opaque and the morphology changes as clearly seen in FIG. 7. The induction time is calculated as the time when nucleation occurs after the droplets have entered the thermodynamically stable p-T region for hydrate formation. All experiments were stopped after 24 h.

Table 2 summarizes the induction time measurements. The reported induction time is the average of at least 25 droplets. The observed sequence of droplet nucleation was random in a spatial and temporal sense, which shows that the experimental approach did not compromise the stochastic nature of nucleation.

TABLE 2

| | | CO₂ | | | | | |
|---|---|---|---|---|---|---|---|
| | | dis- | | | | | |
| | | solution | Droplet | Nucleation | Induction Time (min) | | |
| | Salt Added to | time | Volume | rate | | Std. | |
| Surface | DI Water | (min) | (μL) | (min⁻¹) | Mean | Dev | Range |
| Aluminum | None | 90 | 10 | 0.0018 | 494.1 | 353.6 | 20- |
| 5052 | | | | | | | 1321 |
| Aluminum | None | 90 | 20 | 0.0032 | 296.6 | 230.7 | 27- |
| 5052 | | | | | | | 1000 |
| Aluminum | None | 90 | 40 | 0.0048 | 194.2 | 163.7 | 8-617 |
| 5052 | | | | | | | |
| Aluminum | None | 1440 | 20 | 0.0019 | 501.7 | 402.7 | 21- |
| 5052 | | | | | | | 1422 |
| Aluminum | 3.5 wt. % NaCl | 90 | 20 | 0.0021 | 453.1 | 405.1 | 8- |
| 5052 | | | | | | | 1567 |
| Stainless Steel | None | 90 | 20 | | No | | |
| (T316SS) | | | | | nucleation | | |
| Stainless Steel | 0.0625-5 wt. % | 90 | 20 | | No | | |
| (T316SS) | AlCl₃ | | | | nucleation | | |
| Stainless Steel | 0.0625-5 wt. % | 90 | 20 | | No | | |
| (T316SS) | Al₂(SO₄)₃ | | | | nucleation | | |
| Stainless Steel | 3.5 wt. % NaCl | 90 | 20 | | No | | |
| (T316SS) | | | | | nucleation | | |
| Copper | None | 90 | 20 | | No | | |
| | | | | | nucleation | | |
| Anodized | None | 90 | 20 | | No | | |
| Aluminum | | | | | nucleation | | |

Takeaways from Table 2 are highlighted ahead. First, nucleation was observed only on the aluminum surface. No nucleation was observed on copper, stainless steel, or anodized aluminum surfaces within 24 h. Induction times with Al showed a stochastic nature and ranged from 8 min to 22 h, with every droplet eventually nucleating.

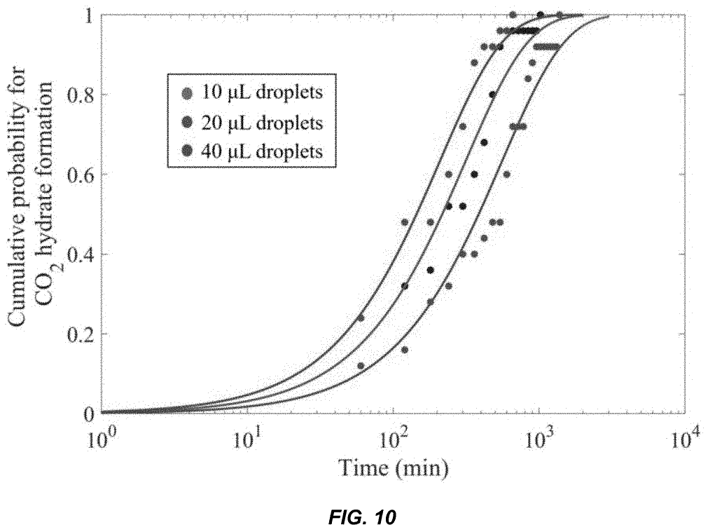

Second, the mean induction time decreased, and the nucleation rate increased with increasing droplet volume. This can be attributed to more nucleation sites becoming available, noting that the three-phase line length and Al-water interfacial area will increase with volume. The similarity between the mean and standard deviation for induction times indicate an underlying exponential distribution. On the basis of classical nucleation theory, the probability (P) for nucleation at a particular subcooling (ΔT=Teq−T) and pressure is given by P(t)=1−exp(−J*t). J is the nucleation rate, which can be obtained by fitting the equation with experimental data. The graph showing droplet volume dependent cumulative probability distribution for nucleation is included shown in FIG. 10.

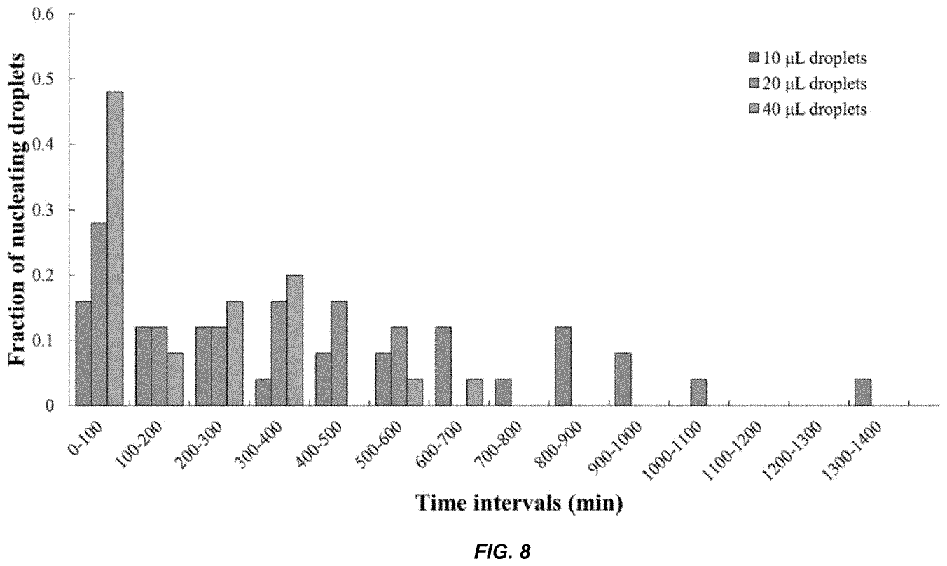

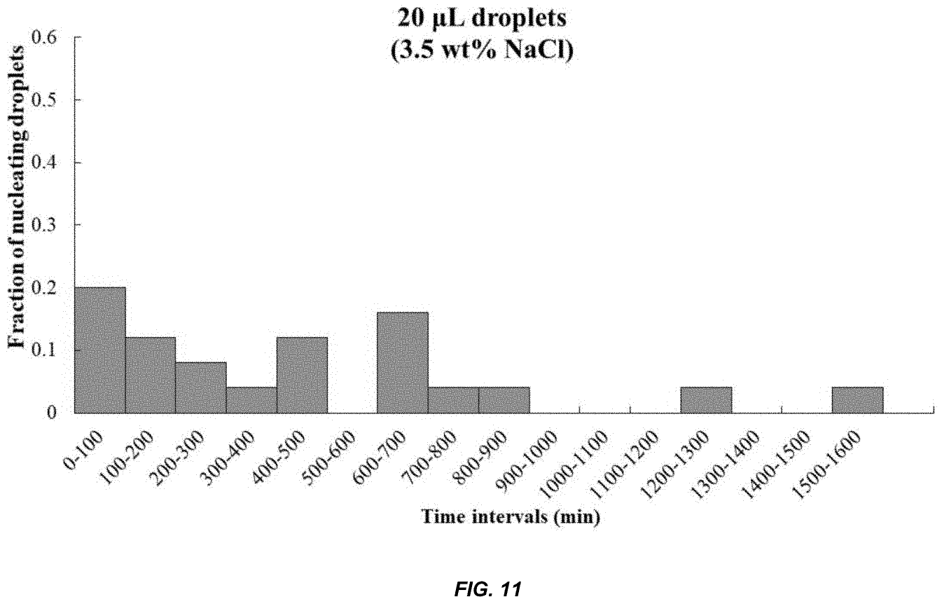

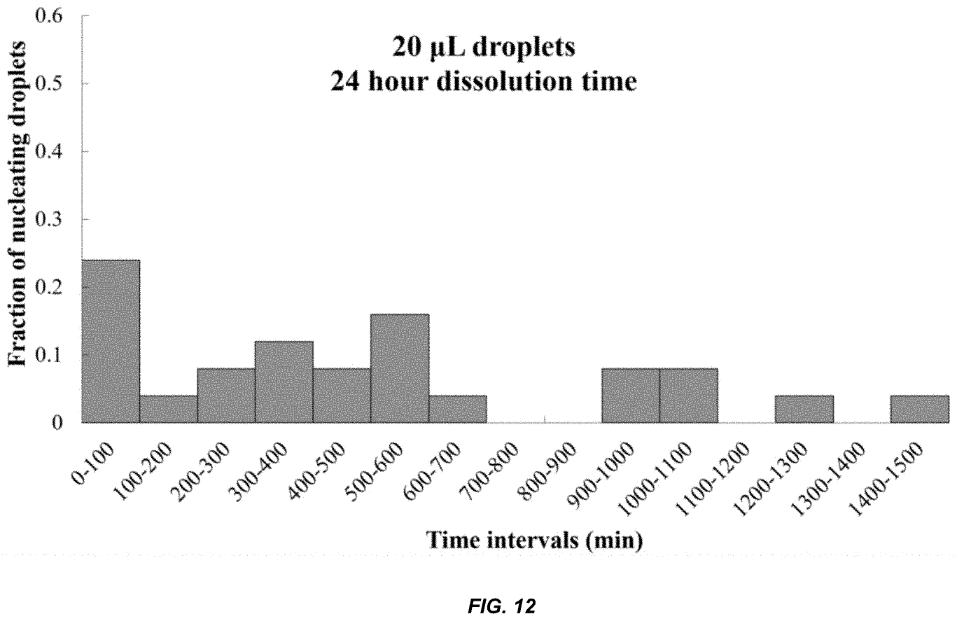

The data on nucleation can be more meaningfully analyzed using a histogram shown in FIG. 8, which shows the fraction of droplets nucleating in different time interval bins for three droplet volumes. It is seen that an increase in the metal-droplet interfacial area (due to increasing droplet volumes) leads to more favorable (faster) nucleation trends. This is reflected in a narrower distribution in the fraction of nucleating droplets, which tends to concentrate toward regions of lower induction time intervals. Additional histograms are shown in FIGS. 11-12.

Third, experiments with water containing 0.6 M sodium chloride (3.5 wt. % NaCl, to mimic seawater concentration), showed a 53% increase in the mean induction time and a 34% reduction in the nucleation rate (20 μL droplets), compared to the results for DI water. This slower nucleation in the presence of salt is consistent with previous observations. Salt ions in aqueous solutions attract water dipoles via Coulombic bonds (much stronger than hydrogen bonding or van der Waals forces), which reduces the availability of water molecules to form hydrates. Importantly, Al surfaces still succeeded in promoting nucleation in saltwater solutions. This repetition of a previously known phenomenon strengthens the scientific rigor of the example approach.

Example results describe the location of the hydrate nucleation sites. Previous studies on hydrate formation report that nucleation may be triggered at the gas-liquid interface due to higher mole fractions of the guest molecule at the interface (at least 2 orders of magnitude higher than the bulk phase). In droplet-based nucleation experiments of hydrates and ice, nucleation is typically observed at the gas-liquid interface or three-phase line, since the nucleation probability is higher than in the other regions of the droplet.

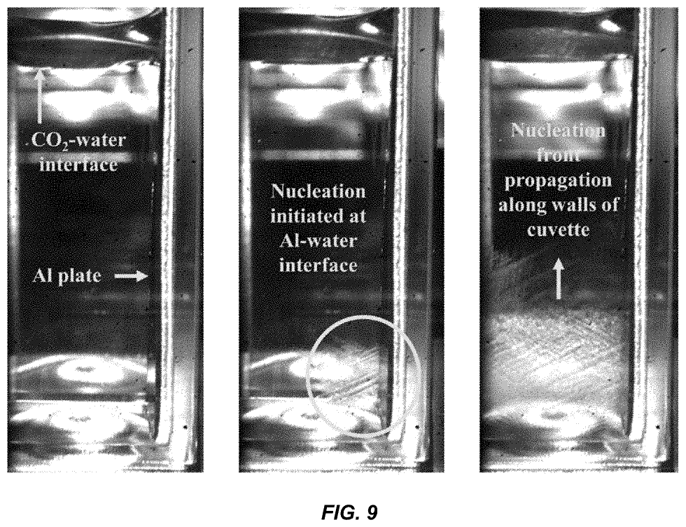

To determine the nucleation sites, experiments were conducted using cuvettes containing 1.75 mL of DI water, with an Al plate dipped as-per two configurations: (i) partially submerged longer plate with a three-phase contact line and (ii) a completely submerged shorter plate without a three-phase line. Baseline experiments were conducted without any plates and with a stainless steel plate; no nucleation was observed. In contrast, nucleation was observed on both configurations of Al plates. Most interestingly, nucleation was consistently initiated at the Al-water interface (in the interior of the liquid), even for the partially submerged plate configuration. This is shown in FIG. 9. This is a very interesting and non-intuitive finding, highlighting the role of the Al-water interface in nucleation promotion.

Example results reveal the influence of aluminum in “catalyzing” nucleation in a CO₂-rich water solution, independent of the gas-phase CO₂. This example reveals nucleation promotion as a consequence of bubbles generated due to reactions at the Al-water interface.

The likely cause of nucleation promotion is hypothesized as not directly related to the ionic species generated at the interface. Instead, hydrogen (H₂) bubble generation at the surface may be responsible for nucleation. It has been reported that hydrogen nanobubbles form at the surface of a platinum electrode; the resulting high Laplace pressure generated due to the small radii leads to favorable conditions for the formation of hydrogen hydrates. Presently, Al reacting with carbonic acid/water will lead to generation of H₂ bubbles, which seed the nucleation of CO₂ hydrates. Carbonic acid will also assist in breaking down any thin or native oxide layers to promote Al-water contact. This hypothesis is also supported by the lack of nucleation on Cu surfaces. Cu is less electropositive than Al and does not easily undergo displacement reactions to generate H₂ which would lead to bubble formation. It is noted that detecting such bubbles visually or via in situ spectroscopy is challenging since these experiments are carried out in a high-pressure cell, and the concentrations of any species will be low. Additional related evidence in support of the proposed mechanism lies in FIG. 8, wherein the increased Al-water interfacial area (and therefore more nucleation sites containing H₂ bubbles) for higher droplet volumes leads to a narrower distribution in the fraction of nucleating droplets. This suggests a causal relation between interfacial area enhancement and nucleation promotion.

Finally, while H₂ bubbles appear to be a probable cause of nucleation promotion, it is likely that there are additional factors such as the roughness/texture at the micro/nano scale that could also promote nucleation.

Figure Captions:

FIG. 6—Schematic illustration of the experimental apparatus.

FIG. 7—Water droplets (left) with a CO₂ dissolution time of 90 min turn opaque (right) upon conversion to CO₂ hydrates (right).

FIG. 8—Histogram showing fraction of droplets nucleating in various time intervals (grouped using 100 min bins) for three different droplet volumes (10, 20, and 40 μL) (dissolution time: 90 min).

FIG. 9—Snapshots depicting CO₂ hydrate nucleation at the Al-water interface (left to right). Nucleation originates at the spot, marked in yellow circle, and proceeds toward the three-phase line.

FIG. 10—Cumulative probability distribution for 3 different droplet volumes (10, 20 and 40 μL).

FIG. 11—Fraction of 20 μL droplets nucleating in different time intervals (droplets contain 3.5 wt. % NaCl).

FIG. 12—Fraction of 20 μL droplets nucleating in different time intervals (24 hour CO₂ dissolution time).

### Example 3: Surfactant Based Promotion of Hydrate Nucleation on Stainless Steel Surfaces

Experimental procedures similar to those used for Examples 1 and 2 were followed. Without Sodium dodecyl-sulfate (SDS), nucleation on stainless steel surfaces did not occur within 40 hours. With SDS, however, nucleation is seen. Nature of nucleation and eventual hydrate is similar to results described in reference to Example 2, above. In drops containing SDS, nucleated drops spread very quickly to induce nucleation in other drops.

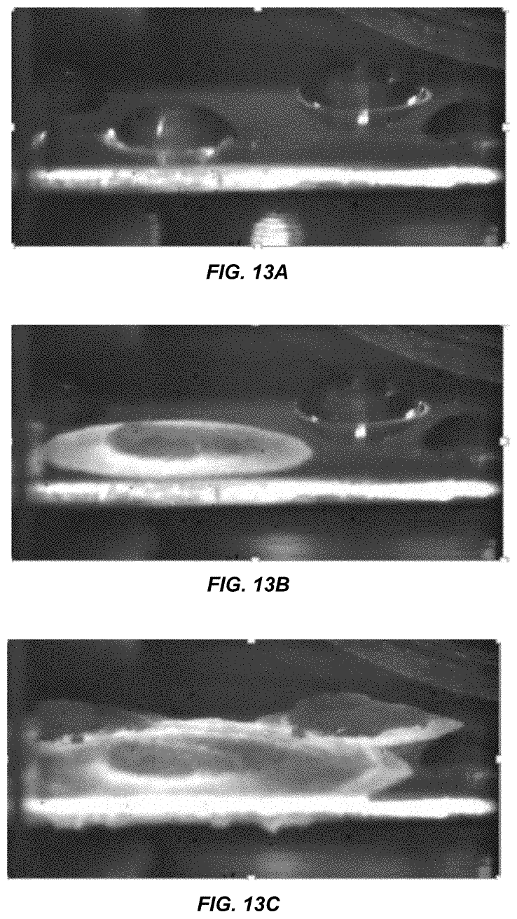

FIG. 13A shows two droplets of water on a stainless steel surface at nucleation conditions, where a first droplet contains SDS.

FIG. 13B shows nucleation in the first droplet containing the SDS, where the first droplet wetted onto the stainless steel surface.

FIG. 13C shows nucleation induced in a second droplet, caused by contact with the first droplet.

Example results are summarized in Table 3. Due to a strong tendency of hydrates to disintegrate during these experiments, only 1 drop was studied at a time. Based on preliminary findings, 3000 ppm SDS in water seems to result in faster nucleation than 2000 ppm SDS in water.

TABLE 3
Nucleation Time of Hydrates on Stainless
Steel for Three Concentrations of SDS

| | 1000 ppm | 2000 ppm | 3000 ppm |
|---|---|---|---|
| Nucleation Time | 20 | 12 | 12 |
| (min) | 108 | 41 | 25 |
| | 116 | 213 | 81 |
| | 309 | 274 | 259 |
| | >24 hrs. | 513 | 378 |
| Mean (min) | 138 | 211 | 151 |
| Std. Dev (min) | 106 | 181 | 144 |

### REFERENCES

Sloan, E. D.; Koh, C. A. Clathrate Hydrates of Natural Gases, 3^{rd} ed.; CRC Press/Taylor-Francis: Boca Raton, Fla., 2007.
Englezos, P. Clathrate Hydrates. Ind. Eng. Chem. Res. 1993, 32 (7), 1251-1274.
Kumar, A.; Bhattacharjee, G.; Kulkarni, B. D.; Kumar, R. Role of Surfactants in Promoting Gas Hydrate Formation. Ind. Eng. Chem. Res. 2015, 54 (49), 12217-12232.
Ganji, H.; Manteghian, M.; Sadaghiani zadeh, K.; Omidkhah, M. R.; Rahimi Mofrad, H. Effect of Different Surfactants on Methane Hydrate Formation Rate, Stability and Storage Capacity. Fuel 2007, 86 (3), 434-441.
Li, A.; Jiang, L.; Tang, S. An Experimental Study on Carbon Dioxide Hydrate Formation Using a Gas-Inducing Agitated Reactor. Energy 2017, 134, 629-637.
Babu, P.; Linga, P.; Kumar, R.; Englezos, P. A Review of the Hydrate Based Gas Separation (HBGS) Process For Carbon Dioxide Pre-Combustion Capture. Energy 2015, 85, 261-279.
Park, T.; Kwon, T.-H. Effect of Electric Field on Gas Hydrate Nucleation Kinetics: Evidence for the Enhanced Kinetics of Hydrate Nucleation by Negatively Charged Clay Surfaces. Environ. Sci. Technol. 2018, 52 (5), 3267-3274.
Acharya, P. V.; Bahadur, V. Fundamental Interfacial Mechanisms Underlying Electrofreezing. Adv. Colloid Interface Sci. 2018, 251, 26-43.
Carpenter, K.; Bahadur, V. Electronucleation for Rapid and Controlled Formation of Hydrates. J. Phys. Chem. Lett. 2016, 7 (13), 2465-2469.
Shahriari, A.; Acharya, P. V.; Carpenter, K.; Bahadur, V. Metal-Foam-Based Ultrafast Electronucleation of Hydrates at Low Voltages. Langmuir 2017, 33 (23), 5652-5656.
Servio, P.; Englezos, P. Morphology of Methane and Carbon Dioxide Hydrates Formed from Water Droplets. AIChE J. 2003, 49 (1), 269-276.
Lee, J. D.; Susilo, R.; Englezos, P. Methane-Ethane and Methane-Propane Hydrate Formation and Decomposition on Water Droplets. Chem. Eng. Sci. 2005, 60 (15), 4203-4212.
Ueno, H.; Akiba, H.; Akatsu, S.; Ohmura, R. Crystal Growth of Clathrate Hydrates Formed with Methane+Carbon Dioxide Mixed Gas at the Gas/Liquid Interface and in Liquid Water. New J. Chem. 2015, 39 (11), 8254-8262.
Saito, K.; Kishimoto, M.; Tanaka, R.; Ohmura, R. Crystal Growth of Clathrate Hydrate at the Interface between Hydrocarbon Gas Mixture and Liquid Water. Cryst. Growth Des. 2011, 11 (1), 295-301.
Servio, P.; Englezos, P. Morphology Study of Structure H Hydrate Formation from Water Droplets. Cryst. Growth Des. 2003, 3 (1), 61-66.
Jeong, K.; Metaxas, P. J.; Chan, J.; Kuteyi, T. O.; Aman, Z. M.; Stanwix, P. L.; Johns, M. L.; May, E. F. Hydrate Nucleation and Growth on Water Droplets Acoustically-Levitated in High-Pressure Natural Gas. Phys. Chem. Chem. Phys. 2019, 21, 21685-21688.
Moudrakovski, I. L.; McLaurin, G. E.; Ratcliffe, C. I.; Ripmeester, J. A. Methane and Carbon Dioxide Hydrate Formation in Water Droplets: Spatially Resolved Measurements from Magnetic Resonance Microimaging. J. Phys. Chem. B 2004.
Carpenter, K.; Bahadur, V. Electrofreezing of Water Droplets under Electrowetting Fields. Langmuir 2015, 31 (7), 2243-2248.
Yang, F.; Shaw, R. A.; Gurganus, C. W.; Chong, S. K.; Yap, Y. K. Ice Nucleation at the Contact Line Triggered by Transient Electrowetting Fields. Appl. Phys. Lett. 2015, 107 (26), 264101.
Kashchiev, D.; Firoozabadi, A. Induction Time in Crystallization of Gas Hydrates. J Cryst. Growth 2003, 250 (3-4), 499-515.
Cussler, E. L. Diffusion: Mass Transfer in Fluid Systems, 2nd ed.; Cambridge university press, 2009.
Rodger, P. M.; Forester, T. R.; Smith, W. Simulations of the Methane Hydrate/Methane Gas Interface near Hydrate Forming Conditions. Fluid Phase Equilib. 1996, 116 (1-2), 326-332.
Khurana, M.; Yin, Z.; Linga, P. A Review of Clathrate Hydrate Nucleation. ACS Sustain. Chem. Eng. 2017, 5 (12), 11176-11203.
Ueno, H.; Akiba, H.; Akatsu, S.; Ohmura, R. Crystal Growth of Clathrate Hydrates Formed with Methane+Carbon Dioxide Mixed Gas at the Gas/Liquid Interface and in Liquid Water. New J. Chem. 2015, 39 (11), 8254-8262.
Hozumi, T.; Saito, A.; Okawa, S.; Watanabe, K. Effects of Electrode Materials on Freezing of Supercooled Water in Electric Freeze Control. Int. J. Refrig. 2003, 26 (5), 537-542.
Hay, M. B.; Myneni, S. C. B. Geometric and Electronic Structure of the Aqueous Al(H₂O)₆³⁺ Complex. J. Phys. Chem. A 2008, 112 (42), 10595-10603.
Hunter, M. S.; Fowle, P. Natural and Thermally Formed Oxide Films on Aluminum. J. Electrochem. Soc. 1956, 103 (9), 482-485.
Kashiwagi, K.; Hattori, T.; Samejima, Y.; Kobayashi, N.; Nakabayashi, S. Hydrogen Nanobubbles at Roughness-Regulated Surfaces: Why Does the Standard Hydrogen Electrode Need a Platinized Platinum Electrode? J. Phys. Chem. C 2019, 123 (12), 7416-7424.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “‘1’ or ‘2’ or ‘3’ or ‘1 and 2’ or ‘1 and 3’ or ‘2 and 3’ or ‘1, 2 and 3’”.

Every formulation or combination of components described or exemplified can be used to practice the invention, unless otherwise stated. Specific names of materials are intended to be exemplary, as it is known that one of ordinary skill in the art can name the same material differently. It will be appreciate that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### ILLUSTRATIVE ASPECTS

As used below, any reference to a series of aspects is to be understood as a reference to each of those aspects disjunctively (e.g., “Aspect(s) 1-4” is to be understood as “Aspects 1, 2, 3, or 4”).

Aspect 1 is a method for generating CO₂ clathrate hydrates, the method comprising: subjecting CO₂ and a liquid comprising water to a clathrate hydrate nucleation condition while contacting the liquid with a reactive metal nucleation substrate, wherein the reactive metal nucleation substrate reacts with the liquid to form a plurality of gas bubbles that facilitate nucleation of a CO₂ clathrate hydrate, and wherein the reactive metal nucleation substrate comprises a Group II element or an alloy thereof, or a Group I element or an alloy thereof, or a Group XIII element or an alloy thereof.

Aspect 2 is the method of aspect 1, wherein the reactive metal nucleation substrate comprises Magnesium or an alloy thereof.

Aspect 3 is the method of aspect(s) 1-2, wherein the reactive metal nucleation substrate comprises Gallium or an alloy thereof.

Aspect 4 is the method of aspect(s) 1-3, wherein the reactive metal nucleation substrate comprises Aluminum or an alloy thereof.

Aspect 5 is the method of aspect(s) 1-4, wherein the reactive metal nucleation substrate comprises Calcium or an alloy thereof.

Aspect 6 is the method of aspect(s) 1-5, wherein the reactive metal nucleation substrate comprises at least one of a dust, a foam, a porous scaffold, a nanostructured material, a coating, a thin film, a plate, a powder, or a felt.

Aspect 7 is the method of aspect(s) 1-6, wherein the reactive metal nucleation substrate comprises a plurality of particles having a diameter of from 10 nm to 100 μm.

Aspect 8 is the method of aspect 7, wherein the plurality of particles are present as a colloidal suspension in the liquid.

Aspect 9 is the method of aspect(s) 1-8, wherein the reactive metal nucleation substrate comprises a scaffold including a plurality of particles, wherein the scaffold includes a void volume such that the liquid flows through the void volume and introduces a plurality of gas bubbles into the liquid.

Aspect 10 is the method of aspect(s) 1-9, wherein the liquid comprises at least one of sea water, fresh water, processed water, purified water, brackish water, hypersaline water, or water including a salt concentration or an ion concentration in a range from 0 to 10% by weight.

Aspect 11 is the method of aspect(s) 1-10, wherein the liquid comprises a dissolved salt comprising at least one of Al₂(SO₄)₃, NaCl, or AlCl₃.

Aspect 12 is the method of aspect(s) 1-11, wherein a total dissolved solids in the liquid is in a range of from 0 and 50,000 ppm.

Aspect 13 is the method of aspect(s) 1-12, wherein the CO₂ comprises at least one of gaseous CO₂, liquid CO₂, or dissolved CO₂.

Aspect 14 is the method of aspect(s) 1-13, wherein the CO₂ has a purity of at least 80%.

Aspect 15 is the method of aspect(s) 1-14, further comprising generating the CO₂ by way of a chemical reaction.

Aspect 16 is the method of aspect(s) 1-15, wherein the liquid comprises a concentration of hydrogen ions represented by a pH value in a range of about 5 to 9.

Aspect 17 is the method of aspect(s) 1-16, wherein the liquid comprises a dissolved CO₂ concentration of from 0 mol/L to 0.15 mol/L.

Aspect 18 is the method of aspect(s) 1-17, further comprising introducing at least one of an additive gas or an additive liquid into the liquid contacting the reactive metal nucleation substrate.

Aspect 19 is the method of aspect 18, wherein the additive gas or the additive liquid comprises CO₂ or a mixture comprising CO₂.

Aspect 20 is the method of aspect(s) 18-19, wherein the additive gas or the additive liquid comprises a promoter for hydrate formation.

Aspect 21 is the method of aspect(s) 18-20, wherein the additive liquid comprises a surfactant or an enzyme.

Aspect 22 is the method of aspect 21, wherein the surfactant comprises at least one of Sodium laureth-sulfate (SLES), Sodium dodecyl-sulfate (SDS), or Ammonium lauryl-sulfate (ALS).

Aspect 23 is the method of aspect(s) 18-22, wherein introducing the additive gas comprises bubbling the additive gas in the liquid or wherein introducing the additive liquid comprises spraying the additive liquid into the liquid.

Aspect 24 is the method of aspect(s) 18-23, wherein introducing the additive gas comprises sparging the additive gas, wherein the sparging provides a plurality of gas bubbles having a diameter in the range from 100 nm to 10 mm.

Aspect 25 is the method of aspect(s) 18-24, wherein introducing the additive gas comprises pre-cooling the additive gas to an introduction temperature below a reactor temperature.

Aspect 26 is the method of aspect(s) 1-25, wherein the clathrate hydrate nucleation condition comprises a pressure of greater than 150 psig or from 150 psig to 4500 psig.

Aspect 27 is the method of aspect(s) 1-26, comprising subjecting the liquid and the CO₂ to the clathrate hydrate nucleation condition in a pressure vessel.

Aspect 28 is the method of aspect 27, further comprising maintaining the CO₂ and the liquid at the clathrate hydrate nucleation condition using a pressure controller in communication with the pressure vessel.

Aspect 29 is the method of aspect(s) 27-28, wherein the pressure vessel comprises a bubble column or an air lift reactor.

Aspect 30 is the method of aspect(s) 1-29, wherein the clathrate hydrate nucleation condition comprises a temperature of from 248 K to 298 K.

Aspect 31 is the method of aspect(s) 1-30, comprising subjecting the CO₂ and the liquid to the clathrate hydrate nucleation condition by removing heat through direct or indirect contact with a heat exchanger.

Aspect 32 is the method of aspect 31, further comprising maintaining the CO₂ and the liquid at the clathrate hydrate nucleation condition using a temperature controller in communication with the heat exchanger.

Aspect 33 is the method of aspect(s) 1-32, wherein nucleation occurs in less than 8 minutes or from about 1 minutes to about 12 minutes after the clathrate hydrate nucleation condition is established.

Aspect 34 is the method of aspect(s) 1-33, wherein the plurality of gas bubbles have a diameter of less than 500 m or from 10 nm to 5 mm.

Aspect 35 is the method of aspect(s) 1-34, wherein the plurality of gas bubbles comprise a reaction product gas, and wherein the reaction product gas comprises hydrogen gas (H₂).

Aspect 36 is the method of aspect(s) 1-35, wherein the plurality of gas bubbles facilitate nucleation in the liquid, at an interface between the liquid and the reactive metal nucleation substrate, or at a gas-liquid-metal interface.

Aspect 37 is the method of aspect(s) 1-36, further comprising inducing convection in the liquid for increasing a clathrate hydrate formation rate.

Aspect 38 is the method of aspect 37, wherein inducing convection in the liquid comprises generating a second plurality of gas bubbles by cavitation in the liquid.

Aspect 39 is the method of aspect(s) 1-38, further comprising separating the CO₂ clathrate hydrate from the liquid.

Aspect 40 is the method of aspect(s) 1-39, further comprising introducing an inert nucleation substrate into the liquid.

Aspect 41 is the method of aspect 40, wherein the inert nucleation substrate comprises sand.

Aspect 42 is the method of aspect(s) 1-41, further comprising subjecting the reactive metal nucleation substrate to a localized nucleation condition.

Aspect 43 is the method of aspect 42, wherein subjecting the reactive metal nucleation substrate to the localized nucleation condition comprises providing ultrasonic acoustic energy to the reactive metal nucleation substrate.

Aspect 44 is the method of aspect(s) 42-43, wherein subjecting the reactive metal nucleation substrate to the localized nucleation condition comprises providing radiant thermal energy to a region of liquid surrounding the reactive metal nucleation substrate by absorption of the radiant thermal energy by a surface of the reactive metal nucleation substrate.

Aspect 45 is the method of aspect(s) 42-44, wherein subjecting the reactive metal nucleation substrate to the localized nucleation condition comprises localized boiling of the liquid at a surface of the reactive metal nucleation substrate.

Aspect 46 is the method of aspect(s) 42-45, wherein subjecting the reactive metal nucleation substrate to the localized nucleation condition comprises generating thermal energy at a surface of the reactive metal nucleation substrate via an exothermic reaction between the reactive metal nucleation substrate and a reactant dissolved in the liquid.

Aspect 47 is the method of aspect(s) 42-46, wherein subjecting the reactive metal nucleation substrate to the localized nucleation condition comprises electrolyzing water to form O₂ and/or H₂.

Aspect 48 is a system for generating CO₂ clathrate hydrates, the system comprising: a vessel comprising a reservoir for subjecting CO₂ and a liquid comprising water to a clathrate hydrate nucleation condition; and a reactive metal nucleation substrate in contact with the liquid, the reactive metal substrate reactive with the liquid to form a plurality of gas bubbles for facilitating nucleation of a CO₂ clathrate hydrate, wherein the reactive metal nucleation substrate comprises a Group II element or an alloy thereof, or a Group I element or an alloy thereof, or a Group XIII element or an alloy thereof.

Aspect 49 is the system of aspect 48, wherein the reactive metal nucleation substrate comprises Magnesium or an alloy thereof.

Aspect 50 is the system of aspect(s) 48-49, wherein the reactive metal nucleation substrate comprises Gallium or an alloy thereof.

Aspect 51 is the system of aspect(s) 48-50, wherein the reactive metal nucleation substrate comprises Aluminum or an alloy thereof.

Aspect 52 is the system of aspect(s) 48-51, wherein the reactive metal nucleation substrate comprises Calcium or an alloy thereof.

Aspect 53 is the system of aspect(s) 48-52, wherein the CO₂ comprises at least one of gaseous CO₂, liquid CO₂, or dissolved CO₂.

Aspect 54 is the system of aspect(s) 48, wherein the CO₂ has a purity of at least 80%.

Aspect 55 is the system of aspect(s) 48-54, further comprising: a pump in fluid communication with the vessel for generating a pressure in the vessel associated with the clathrate hydrate nucleation condition.

Aspect 56 is the system of aspect 55, further comprising: a pressure controller in fluid communication with the vessel and in control communication with the pump for controlling the pressure in the vessel.

Aspect 57 is the system of aspect(s) 48-56, further comprising: a heat exchanger in thermal communication with the vessel for generating a temperature in the vessel associated with the clathrate hydrate nucleation.

Aspect 58 is the system of aspects 57, further comprising: a temperature controller in thermal communication with the vessel and in control communication with the heat exchanger for controlling the temperature in the vessel.

Aspect 59 is the system of aspect(s) 48-58, wherein the vessel comprises a bubble column or an air lift reactor.

Aspect 60 is the system of aspect(s) 48-59, further comprising: one or more processors; and a non-transitory computer readable storage medium in communication with the one or more processors, the non-transitory computer readable storage medium containing instructions that, when executed by the one or more processors, cause the one or more processors to perform operations including: controlling or maintaining a pressure in the vessel associated with the clathrate hydrate nucleation condition by receiving pressure sensor measurements and sending a pressure control signal to a pump in fluid communication with the vessel; or controlling or maintaining a temperature in the vessel associated with the clathrate hydrate nucleation condition by receiving temperature sensor measurements and sending a temperature control signal to a heat exchanger in thermal communication with the vessel.

Aspect 61 is the system of aspect(s) 48-60, wherein the reactive metal nucleation substrate comprises at least one of a dust, a foam, a porous scaffold, a nanostructured material, a coating, a thin film, a plate, a powder, or a felt.

Aspect 62 is the system of aspect(s) 48-61, wherein the reactive metal nucleation substrate comprises a plurality of particles having a diameter of 10 nm to 100 m in a colloidal suspension in the liquid.

Aspect 63 is the system of aspect(s) 48-62, wherein the reactive metal nucleation substrate comprises a scaffold including a plurality of particles including a Group II metal, wherein the scaffold includes a void volume such that the liquid flows through the void volume and introduces the plurality of gas bubbles into the liquid.

Aspect 64 is the system of aspect(s) 48-63, wherein the liquid comprises at least one of sea water, fresh water, processed water, purified water, brackish water, hypersaline water, or water including a salt concentration or an ion concentration in a range from 0 to 10% by weight.

Aspect 65 is the system of aspect(s) 48-64, wherein the liquid comprises a dissolved salt comprising at least one of Al₂(SO₄)₃, NaCl, or AlCl₃.

Aspect 66 is the system of aspect(s) 48-65, wherein a total dissolved solids in the liquid is in a range of from 0 and 50,000 ppm.

Aspect 67 is the system of aspect(s) 48-66, wherein the liquid comprises a concentration of hydrogen ions represented by a pH value in a range of about 5 to 9.

Aspect 68 is the system of aspect(s) 48-67, wherein the liquid comprises a dissolved CO₂ concentration of from 0 mol/L to 0.15 mol/L.

Aspect 69 is the system of aspect(s) 48-68, wherein the liquid comprises a promoter for hydrate formation.

Aspect 70 is the system of aspect(s) 48-69, wherein the liquid comprises a surfactant or an enzyme.

Aspect 71 is the system of aspect 70, wherein the surfactant comprises at least one of Sodium laureth-sulfate (SLES), Sodium dodecyl-sulfate (SDS), or Ammonium lauryl-sulfate (ALS).

Aspect 72 is the system of aspect(s) 48-71, wherein the clathrate hydrate nucleation condition comprises a pressure of greater than 150 psig or from 150 psig to 4500 psig.

Aspect 73 is the system of aspect(s) 48-72, wherein the clathrate hydrate nucleation condition comprises a temperature of from 248 K to 298 K.

Aspect 74 is the system of aspect(s) 48-73, wherein the vessel is configured to maintain the liquid and the gas-phase CO₂ or CO₂ dissolved in the liquid at the clathrate hydrate nucleation condition for a period of time until an onset of CO₂ clathrate hydrate formation.

Aspect 75 is the system of aspect 74, wherein the period of time is less than 8 minutes or from about 1 minute to about 12 minutes.

Aspect 76 is the system of aspect(s) 48-75, further comprising an optical sensor configured to generate images of an interior region of the vessel.

Aspect 77 is a method for generating clathrate hydrates, the method comprising: subjecting a compound and liquid comprising water to a clathrate hydrate nucleation condition while forming a plurality of gas bubbles that facilitate nucleation of a clathrate hydrate comprising water and the compound, wherein the compound is in a gaseous state, a liquid state, or is dissolved in the liquid; and maintaining the compound and the liquid at the clathrate hydrate nucleation condition for a period of time until an onset of clathrate hydrate nucleation, wherein the period of time is less than 8 minutes or is from about 1 minutes to about 12 minutes.

Aspect 78 is the method of aspect(s) 77, wherein the liquid comprises at least one of sea water, fresh water, processed water, purified water, brackish water, hypersaline water, or water including a salt or ion concentration in a range from 0 to 30% by weight.

Aspect 79 is the method of aspect(s) 77-78, wherein the compound comprises at least one of CO₂, methane, ethane, propane, butane, hydrogen, tetrahydrofuran, or cyclopentane.

Aspect 80 is the method of aspect(s) 77-79, wherein forming the plurality of gas bubbles comprises contacting the liquid with a reactive metal nucleation substrate, and wherein the reactive metal nucleation substrate comprises a Group II element or an alloy thereof, or a Group I element or an alloy thereof, or a Group XIII element or an alloy thereof.

Aspect 81 is the method of aspect 80, wherein the reactive metal nucleation substrate comprises Magnesium or an alloy thereof.

Aspect 82 is the method of aspect(s) 80-81, wherein the reactive metal nucleation substrate comprises Gallium or an alloy thereof.

Aspect 83 is the method of aspect(s) 80-82, wherein the reactive metal nucleation substrate comprises Aluminum or an alloy thereof.

Aspect 84 is the method of aspect(s) 80-83, wherein the reactive metal nucleation substrate comprises Calcium or an alloy thereof.

Aspect 85 is the method of aspect(s) 80-84, wherein the reactive metal nucleation substrate comprises at least one of a dust, a foam, a porous scaffold, a nanostructured material, a coating, a thin film, a plate, a powder, or a felt.

Aspect 86 is the method of aspect(s) 80-85, wherein the reactive metal nucleation substrate comprises a plurality of particles having a diameter of 10 nm to 100 m.

Aspect 87 is the method of aspect 86, wherein the plurality of particles are present as a colloidal suspension in the liquid.

Aspect 88 is the method of aspect(s) 80-87, wherein the reactive metal nucleation substrate comprises a scaffold including a plurality of particles including a Group II metal, wherein the scaffold includes a void volume such that the liquid flows through the void volume and introduces the plurality of gas bubbles into the liquid.

Aspect 89 is the method of aspect(s) 77-88, wherein the clathrate hydrate nucleation condition comprises a pressure of greater than 150 psig or from 150 psig to 4500 psig.

Aspect 90 is the method of aspect(s) 77-89, wherein the clathrate hydrate nucleation condition comprises a temperature of from 248 K to 298 K.

Aspect 91 is the method of aspect(s) 77-90, wherein forming the plurality of gas bubbles comprises applying ultrasonic energy to the liquid.

Aspect 92 is the method of aspect(s) 77-91, wherein forming the plurality of gas bubbles comprises electrolyzing water to form O₂ and/or H₂.

Aspect 93 is the method of aspect(s) 77-92, wherein the compound is CO₂ and is dissolved in the liquid at a concentration of from 0 mol/L to 0.15 mol/L.

Aspect 94 is the method of aspect(s) 77-93, wherein the compound has a purity of at least 80%.

Aspect 95 is the method of aspect(s) 77-94, wherein the plurality of gas bubbles having a diameter of less than 500 m or from 10 nm to 5 mm.

Aspect 96 is the method of aspect(s) 77-95, wherein the plurality of gas bubbles facilitate nucleation in the liquid, at an interface between the liquid and the reactive metal nucleation substrate, or at a gas-liquid-metal interface.

Aspect 97 is the method of aspect(s) 77-96, further comprising inducing convection in the liquid for increasing a clathrate hydrate formation rate.

Aspect 98 is the method of aspect(s) 77-97, further comprising separating the clathrate hydrate from the liquid.

Aspect 99 is the method of aspect(s) 77-98, further comprising introducing at least one of an additive gas or an additive liquid into the liquid contacting the reactive metal nucleation substrate.

Aspect 100 is the method of aspect 99, wherein the additive gas or the additive liquid comprises CO₂ or a mixture comprising CO₂.

Aspect 101 is the method of aspect(s) 99-100, wherein the additive gas or the additive liquid comprises a promoter for hydrate formation.

Aspect 102 is the method of aspect(s) 99-101, wherein the additive liquid comprises a surfactant or an enzyme.

Aspect 103 is the method of aspect(s) 102, wherein the surfactant comprises at least one of Sodium laureth-sulfate (SLES), Sodium dodecyl-sulfate (SDS), or Ammonium lauryl-sulfate (ALS).

Aspect 104 is the method of aspect(s) 99-103, wherein introducing the additive gas comprises bubbling the additive gas in the liquid or wherein introducing the additive liquid comprises spraying the additive liquid into the liquid.

Aspect 105 is the method of aspect(s) 99-104, wherein introducing the additive gas comprises sparging the additive gas, wherein the sparging provides a plurality of gas bubbles having a diameter in the range from 100 nm to 10 mm.

Aspect 106 is the method of aspect(s) 99-105, wherein introducing the additive gas comprises pre-cooling the additive gas to an introduction temperature below an ambient temperature.

1. A method for generating CO₂ clathrate hydrates, the method comprising:
subjecting CO₂ and a liquid comprising water to a clathrate hydrate nucleation condition while contacting the liquid with a reactive metal nucleation substrate, wherein the reactive metal nucleation substrate reacts with the liquid to form a plurality of gas bubbles that facilitate nucleation of a CO₂ clathrate hydrate, and wherein the reactive metal nucleation substrate comprises a Group II element or an alloy thereof, or a Group I element or an alloy thereof, or a Group XIII element or an alloy thereof.

2. The method of claim 1, wherein the reactive metal nucleation substrate comprises Magnesium or an alloy thereof.

3. The method of claim 1, wherein the reactive metal nucleation substrate comprises Gallium or an alloy thereof, Aluminum or an alloy thereof, or Calcium or an alloy thereof.

4.-5. (canceled)

6. The method of claim 1, wherein the reactive metal nucleation substrate comprises at least one of a dust, a foam, a porous scaffold, a nanostructured material, a coating, a thin film, a plate, a powder, or a felt.

7.-9. (canceled)

10. The method of claim 1, wherein the liquid comprises at least one of sea water, fresh water, processed water, purified water, brackish water, hypersaline water, or water including a salt concentration or an ion concentration in a range from 0 to 10% by weight.

11.-12. (canceled)

13. The method of claim 1, wherein the CO₂ comprises at least one of gaseous CO₂, liquid CO₂, or dissolved CO₂.

14.-17. (canceled)

18. The method of claim 1, further comprising introducing at least one of an additive gas or an additive liquid into the liquid contacting the reactive metal nucleation substrate, wherein the additive gas or the additive liquid comprises one or more of a promoter for hydrate formation, a surfactant, or an enzyme.

19.-24. (canceled)

25. The method of claim 18, wherein introducing the additive gas comprises pre-cooling the additive gas to an introduction temperature below a reactor temperature.

26. The method of claim 1, wherein the clathrate hydrate nucleation condition comprises a pressure of greater than 150 psig or from 150 psig to 4500 psig.

27. The method of claim 1, comprising subjecting the liquid and the CO₂ to the clathrate hydrate nucleation condition in a pressure vessel, wherein the pressure vessel comprises a bubble column reactor or an air lift reactor.

28.-32. (canceled)

33. The method of claim 1, wherein nucleation occurs in less than 8 minutes or from about 1 minutes to about 12 minutes after the clathrate hydrate nucleation condition is established.

34. (canceled)

35. The method of claim 1, wherein the plurality of gas bubbles comprise a reaction product gas, and wherein the reaction product gas comprises hydrogen gas (H₂).

36. The method of claim 1, wherein the plurality of gas bubbles facilitate nucleation in the liquid, at an interface between the liquid and the reactive metal nucleation substrate, or at a gas-liquid-metal interface.

37.-47. (canceled)

48. A system for generating CO₂ clathrate hydrates, the system comprising:
a vessel comprising a reservoir for subjecting CO₂ and a liquid comprising water to a clathrate hydrate nucleation condition; and
a reactive metal nucleation substrate in contact with the liquid, the reactive metal substrate reactive with the liquid to form a plurality of gas bubbles for facilitating nucleation of a CO₂ clathrate hydrate, wherein the reactive metal nucleation substrate comprises a Group II element or an alloy thereof, or a Group I element or an alloy thereof, or a Group XIII element or an alloy thereof.

49.-54. (canceled)

55. The system of claim 48, further comprising one or more of:
a pump in fluid communication with the vessel for generating a pressure in the vessel associated with the clathrate hydrate nucleation condition;
a pressure controller in fluid communication with the vessel and in control communication with the pump for controlling the pressure in the vessel;
a heat exchanger in thermal communication with the vessel for generating a temperature in the vessel associated with the clathrate hydrate nucleation;
a temperature controller in thermal communication with the vessel for controlling or monitoring a temperature in the vessel.

56.-59. (canceled)

60. The system of claim 48, further comprising:
one or more processors; and
a non-transitory computer readable storage medium in communication with the one or more processors, the non-transitory computer readable storage medium containing instructions that, when executed by the one or more processors, cause the one or more processors to perform operations including:
controlling or maintaining a pressure in the vessel associated with the clathrate hydrate nucleation condition by receiving pressure sensor measurements and sending a pressure control signal to a pump in fluid communication with the vessel; or
controlling or maintaining a temperature in the vessel associated with the clathrate hydrate nucleation condition by receiving temperature sensor measurements and sending a temperature control signal to a heat exchanger in thermal communication with the vessel.

61.-76. (canceled)

77. A method for generating clathrate hydrates, the method comprising:
subjecting a compound and liquid comprising water to a clathrate hydrate nucleation condition while forming a plurality of gas bubbles that facilitate nucleation of a clathrate hydrate comprising water and the compound, wherein the compound is in a gaseous state, a liquid state, or is dissolved in the liquid; and
maintaining the compound and the liquid at the clathrate hydrate nucleation condition for a period of time until an onset of clathrate hydrate nucleation, wherein the period of time is less than 8 minutes or is from about 1 minutes to about 12 minutes.

78. (canceled)

79. The method of claim 77, wherein the compound comprises at least one of CO₂, methane, ethane, propane, butane, hydrogen, tetrahydrofuran, or cyclopentane.

80. The method of claim 77, wherein forming the plurality of gas bubbles comprises contacting the liquid with a reactive metal nucleation substrate, and wherein the reactive metal nucleation substrate comprises a Group II element or an alloy thereof, or a Group I element or an alloy thereof, or a Group XIII element or an alloy thereof.

81.-90. (canceled)

91. The method of claim 77, wherein forming the plurality of gas bubbles comprises applying ultrasonic energy to the liquid.

92. The method of claim 77, wherein forming the plurality of gas bubbles comprises electrolyzing water to form O₂ and/or H₂.

93-106. (canceled)
